(19) United States
(12) Patent Application Publication
(10) Pub. No.: US 2004/0221400 A1
(43) Pub. Date: Nov. 11, 2004

(54) Dyeing composition comprising a cationic tertiary para-phenylenediamine and a monosaccharide or disaccharide, processes and uses

(75) Inventors: Jean Cotteret, Verneuil Sur Seine (FR); Alain Lagrange, Coupvray (FR)

Correspondence Address: Mark B. Wilson, Fulbright & Jaworski L.L.P., Suite 2400, 600 Congress Avenue, Austin, TX, 78701, US

(73) Assignee: L'OREAL, Paris (FR)

(21) Appl. No.: 10/735,292

(22) Filed: Dec. 12, 2003

Related U.S. Application Data
(60) Provisional application No. 60/444,623, filed on Feb. 4, 2003.

(30) Foreign Application Priority Data
Dec. 13, 2002 (FR) 02/15775

(51) Int. Cl. A61K 7/13

(52) U.S. Cl. 8/405

(57) ABSTRACT

The present application relates to a composition for the dyeing of keratin fibres, particularly human keratin fibres such as hair, comprising, in an appropriate dyeing medium, at least one cationic tertiary paraphenylenediamine containing a pyrrolidine ring, and at least one particular carbohydrate selected from monosaccharides, disaccharides and mixtures thereof.

The invention further relates to the dyeing process in which this composition is used.

### CROSS REFERENCE TO RELATED APPLICATIONS

This application claims priority to French Application No. 02/15775 filed 13 Dec. 2003, and further claims the benefit of U.S. Provisional Application No. 60/444,623 filed 04 Feb. 2003, the entire disclosures of which are incorporated herein by reference in their entirety.

### BACKGROUND OF THE INVENTION

The present application relates to a composition for the dyeing of keratin fibres, particularly human keratin fibres such as hair, comprising, in an appropriate dyeing medium, at least one cationic tertiary paraphenylenediamine containing a pyrrolidine ring, and at least one particular carbohydrate selected from monosaccharides, disaccharides and mixtures thereof.

The invention further relates to the use of this composition for the dyeing of keratin fibres and to the dyeing process in which this composition is used.

It is known to dye keratin fibres, and particularly human hair, with dyeing compositions containing oxidation dyestuff precursors, generally known as oxidation bases, such as ortho- or paraphenylenediamines, ortho- or paraminophenols and heterocyclic compounds. These oxidation bases are colourless or weakly coloured compounds which, when combined with oxidizing products, can give rise to coloured compounds via an oxidative condensation process.

It is also known that the shades obtained with these oxidation bases can be varied by combining them with couplers or colouration modifiers, the latter being selected especially from aromatic metadiamines, metaminophenols, metadiphenols and certain heterocyclic compounds such as indole compounds.

The variety of molecules employed as oxidation bases and couplers enables a rich palette of colours to be obtained.

Moreover, the so-called “permanent” colouration obtained by virtue of these oxidation dyestuffs must satisfy a number of requirements. Thus it must have no drawbacks from the toxicological point of view, it must afford shades in the desired intensity and it must have a good resistance to external agents such as light, adverse weather conditions, washing, perming, perspiration and rubbing.

The dyestuffs must also be capable of covering white hair and, finally, be as unselective as possible, i.e. afford the smallest possible differences in colouration along the entire length of one and the same keratin fibre, which is generally sensitized (i.e. damaged) to different extents between its tip and its root.

Patent application WO 02/45675 has already proposed the use, for keratin fibres, of oxidation dyeing compositions comprising a cationic tertiary paraphenylenediamine containing a pyrrolidine ring.

These cationic tertiary paraphenylenediamines containing a pyrrolidine ring produce compositions whose safety is generally considered to be better than that of compositions containing conventional paraphenylenediamines. However, the shades obtained when using these compositions are appreciably less intense and appreciably more selective, i.e. the dyeings obtained exhibit substantial variations in colouration according to the degree of sensitization of the different strands of hair or the different zones of one and the same strand of hair. The persistence of these shades can also vary considerably according to the degree of sensitization. In addition, the colourations obtained are often greyer, i.e. less chromatic.

### SUMMARY OF THE INVENTION

Surprisingly and advantageously, the Applicant has now discovered that it is possible to obtain novel compositions for the dyeing of keratin fibres, particularly human keratin fibres such as hair, which are capable of overcoming the disadvantages mentioned above and, in particular, of producing colourations with shades that are varied, chromatic, intense, aesthetic and relatively unselective and that have a good resistance to the various aggressions to which the fibres can be subjected, by combining at least one cationic tertiary paraphenylenediamine containing a pyrrolidine ring, and at least one particular carbohydrate, in one composition. In addition, these compositions have a good toxicological profile.

The invention further relates to a dyeing process in which this composition is used, and to a multicompartment dyeing device, or dyeing kit.

The invention further relates to the use of the composition of the present invention for the dyeing of keratin fibres, particularly human keratin fibres such as hair.

The composition of the present invention makes it possible in particular to obtain a keratin fibre colouration that is chromatic, very intense, relatively unselective and persistent, while at the same time avoiding degradation of these fibres.

In terms of the present invention, cationic tertiary paraphenylenediamine containing a pyrrolidine ring is understood as meaning a paraphenylenediamine possessing an NH₂ group and, in the para position thereto, a disubstituted amine group whose substituents form a pyrrolidine ring with the nitrogen atom, the molecule possessing at least one quaternized nitrogen atom.

Within the framework of the present invention, alkyl is understood as meaning a linear or branched radical, for example methyl, ethyl, n-propyl, isopropyl, butyl, etc. An alkoxy radical is a radical alk-O, the alkyl radical being as defined above. Halogen is preferably Cl, Br, I or F.

The compounds of formula (I) below, and their addition salts, may be mentioned in particular among the cationic tertiary paraphenylenediamines containing a pyrrolidine ring which can be used in the composition according to the present invention:

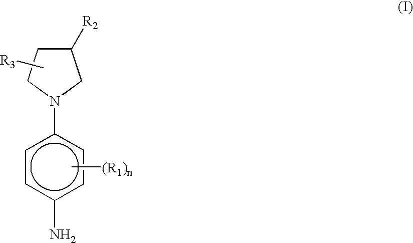

in which:

n varies from 0 to 4, it being understood that if n is greater than or equal to 2, the radicals R₁ can be identical or different;

R₁ is a halogen atom; a saturated or unsaturated, aliphatic or alicyclic C₁-C₆ hydrocarbon chain, it being possible for the chain to contain one or more oxygen, nitrogen, silicon or sulphur atoms or an SO₂ group and to be substituted by one or more hydroxyl or amino radicals; or an onium radical Z, the radical R₁ containing neither a peroxide linkage nor diazo, nitro or nitroso radicals;

R₂ is an onium radical Z or a radical —X—C═NR₈—NR₉R₁₀, in which X is an oxygen atom or a radical —NR₁₁, and R₈, R₉, R₁₀ and R₁₁ are a hydrogen atom, a C₁-C₄ alkyl radical or a C₁-C₄ hydroxyalkyl radical; and

R₃ is a hydrogen atom or a hydroxyl radical.

Onium denotes a quaternary radical of a nitrogen base.

By way of example, R₁ can be a chlorine atom or a methyl, ethyl, isopropyl, vinyl, allyl, methoxymethyl, hydroxyethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy or 2-hydroxyethoxy radical.

In particular, n is equal to 0.

In formula (I), if n is equal to 1, R₁ is preferably a halogen atom or a saturated or unsaturated, aliphatic or alicyclic C₁-C₆ hydrocarbon chain, it being possible for one or more carbon atoms to be replaced by an oxygen, nitrogen, silicon or sulphur atom or by an SO₂ group, the radical R₁ containing neither a peroxide linkage nor diazo, nitro or nitroso radicals. Preferably, R₁ is selected from chlorine, bromine and C₁-C₄ alkyl, C₁-C₄ hydroxyalkyl, C₁-C₄ aminoalkyl, C₁-C₄ alkoxy and C₁-C₄ hydroxyalkoxy radicals. By way of example, R₁ is selected from methyl, hydroxymethyl, 2-hydroxyethyl, 1,2-dihydroxyethyl, methoxy, isopropoxy and 2-hydroxyethoxy radicals.

In one particular embodiment, the radical R₂ in formula (I) is the onium radical Z of formula (II):

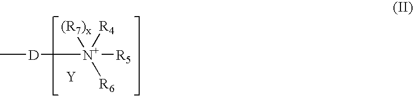

in which:

D is a single bond or a linear or branched C₁-C₁₄ alkylene chain capable of containing one or more heteroatoms selected from oxygen, sulphur and nitrogen, capable of being substituted by one or more hydroxyl, C₁-C₆ alkoxy or amino radicals and capable of carrying one or more ketone groups;

R₄, R₅ and R₆, taken separately, are a C₁-C₁₅ alkyl radical; a C₁-C₆ monohydroxyalkyl radical; a C₂-C₆ polyhydroxyalkyl radical; a C₁-C₆ alkoxy(C₁-C₆)-alkyl radical; an aryl radical; a benzyl radical; a C₁-C₆ amidoalkyl radical; a C₁-C₆ trialkyl(C₁-C₆)silanalkyl radical; a C₁-C₆ aminoalkyl radical; or a C₁-C₆ aminoalkyl radical in which the amine is monosubstituted or disubstituted by a C₁-C₄ alkyl, alkyl(C₁-C₆)carbonyl, amido or alkyl(C₁-C₆)sulphonyl radical; or

R₄, R₅ and R₆, taken together in pairs, form, with the nitrogen atom to which they are attached, a 4-, 5-, 6- or 7-membered saturated carbon-containing ring capable of containing one or more heteroatoms, for example an azetidine ring, a pyrrolidine ring, a piperidine ring, a piperazine ring or a morpholine ring, it being possible for the cationic ring to be substituted by a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a C₁-C₆ trialkyl(C₁-C₆)silanalkyl radical, an amido radical, a carboxyl radical, an alkyl(C₁-C₆)carbonyl radical, a thio radical (—SH), a C₁-C₆ thioalkyl radical (—R—SH), an alkyl(C₁-C₆)thio radical, an amino radical or an amino radical monosubstituted or disubstituted by an alkyl(C₁-C₆), alkyl(C₁-C₆)carbonyl, amido or alkyl(C₁-C₆)sulphonyl radical;

R₇ is a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carboxyalkyl radical; a C₁-C₆ carbamylalkyl radical; a C₁-C₆ trifluoroalkyl radical; a C₁-C₆ trialkyl(C₁-C₆)silanalkyl radical; a C₁-C₆ sulphonamidoalkyl radical; a C₁-C₆ alkyl(C₁-C₆)carboxyalkyl radical; a C₁-C₆ alkyl(C₁-C₆)sulphinylalkyl radical; a C₁-C₆ alkyl(C₁-C₆)sulphonylalkyl radical; a C₁-C₆ alkyl(C₁-C₆)carbonylalkyl radical; a C₁-C₆ N-alkyl(C₁-C₆)carbamylalkyl radical; or a C₁-C₆ N-alkyl(C₁-C₆)sulphonamidoalkyl radical;

x is 0 or 1:

if x=0, the linking arm is attached to the nitrogen atom carrying the radicals R₄ to R₆;

if x=1, two of the radicals R₄ to R₆ form a 4-, 5-, 6- or 7-membered saturated ring together with the nitrogen atom to which they are attached, and D is bonded to a carbon atom of the saturated ring; and

Y is a counterion.

In formula (II), if x is equal to 0, R₄, R₅ and R₆, taken separately, are preferably selected from a C₁-C₆ alkyl radical; a C₁-C₄ monohydroxyalkyl radical; a C₂-C₄ polyhydroxyalkyl radical; a C₁-C₄ alkoxy(C₁-C₆)alkyl radical; a C₁-C₆ amidoalkyl radical; and a C₁-C₆ trialkyl(C₁-C₆)silanalkyl radical, or R₄ and R₅ together form an azetidine, pyrrolidine, piperidine, piperazine or morpholine ring, R6 being selected in this case from a C₁-C₆ alkyl radical; a C₁-C₆ monohydroxyalkyl radical; a C₂-C₆ polyhydroxyalkyl radical; a C₁-C₆ aminoalkyl radical; an aminoalkyl radical monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carboxyalkyl radical; a C₁-C₆ alkyl(C₁-C₆)carbonylalkyl radical; and a C₁-C₆ N-alkyl(C₁-C₆)carbamylalkyl radical.

If x is equal to 1, R₇ is preferably selected from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carboxyalkyl radical; a C₁-C₆ alkyl(C₁-C₆)carbonylalkyl radical; and a C₁-C₆ N-alkyl(C₁-C₆)carbamylalkyl radical; and R₄ and R₅ together form an azetidine, pyrrolidine, piperidine, piperazine or morpholine ring, R₆ being selected in this case from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carboxyalkyl radical; a C₁-C₆ alkyl(C₁-C₆)carbonylalkyl radical; and a C₁-C₆ N-alkyl(C₁-C₆)carbamylalkyl radical.

In formula (II), D is preferably a single bond or an alkylene chain capable of being substituted.

If the radical R₂ has formula (II), it is preferably a trialkylammonium radical whose alkyl radicals can be substituted.

In a second embodiment, the radical R₂ is the onium radical Z of formula (III):

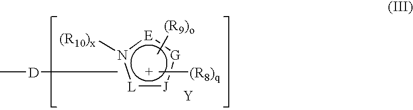

in which:

D is a single bond or a linear or branched C₁-C₁₄ alkylene chain capable of containing one or more heteroatoms selected from oxygen, sulphur and nitrogen, capable of being substituted by one or more hydroxyl, C₁-C₆ alkoxy or amino radicals and capable of carrying one or more ketone functional groups;

the vertices E, G, J and L, which are identical or different, are a carbon, oxygen, sulphur or nitrogen atom so as to form a pyrrole, pyrazole, imidazole, triazole, oxazole, isooxazole, thiazole or isothiazole ring;

q is an integer between 0 and 4 inclusive;

o is an integer between 0 and 3 inclusive;

q+o is an integer between 0 and 4;

the radicals R₈, which are identical or different, are a halogen atom; a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a carboxyl radical; a C₁-C₆ alkylcarbonyl radical; a thio radical; a C₁-C₆ thioalkyl radical; an alkyl(C₁-C₆)thio radical; an amino radical; an amino radical monosubstituted or disubstituted by an alkyl(C₁-C₆), alkyl(C₁-C₆)carbonyl, amido or alkyl(C₁-C₆)sulphonyl radical; a C₁-C₆ monohydroxyalkyl radical; or a C₂-C₆ polyhydroxyalkyl radical, it being understood that the radicals R₈ are carried by a carbon atom;

the radicals R₉, which are identical or different, are a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; a C₁-C₆ carbamylalkyl radical; a C₁-C₆ alkyl(C₁-C₆)carboxyalkyl radical; or a benzyl radical, it being understood that the radicals R₉ are carried by a nitrogen;

R₁₀ is a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is substituted by an alkyl(C₁-C₆), alkyl(C₁-C₆)carbonyl, amido or alkyl(C₁-C₆)sulphonyl radical; a C₁-C₆ carboxyalkyl radical; a C₁-C₆ carbamylalkyl radical; a C₁-C₆ trifluoroalkyl radical; a C₁-C₆ trialkyl(C₁-C₆)silanalkyl radical; a C₁-C₆ sulphonamidoalkyl radical; a C₁-C₆ alkyl(C₁-C₆)carboxyalkyl radical; a C₁-C₆ alkyl(C₁-C₆)sulphinylalkyl radical; a C₁-C₆ alkyl(C₁-C₆)sulphonylalkyl radical; a C₁-C₆ alkyl(C₁-C₆)carbonylalkyl radical; a C₁-C₆ N-alkyl(C₁-C₆)carbarnylalkyl radical; or a C₁-C₆ N-alkyl(C₁-C₆)sulphonamidoalkyl radical;

x is 0 or 1:

if x=0, the linking arm D is attached to the nitrogen atom;

if x=1, the linking arm D is attached to one of the vertices E, G, J or L; and

Y is a counterion.

The vertices E, G, J and L preferably form an imidazole ring.

Among the radicals R₂ of formula (III), preference is given to the radicals in which x is equal to 0 and D is a single bond or an alkylene chain capable of being substituted.

In a third embodiment, R₂ is the onium radical Z of formula (IV):

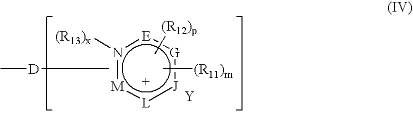

in which:

D is a single bond or a linear or branched C₁-C₁₄ alkylene chain capable of containing one or more heteroatoms selected from oxygen, sulphur and nitrogen atoms, capable of being substituted by one or more hydroxyl, C₁-C₆ alkoxy or amino radicals and capable of carrying one or more ketone functional groups;

the vertices E, G, J, L and M, which are identical or different, are a carbon, oxygen, sulphur or nitrogen atom so as to form a ring selected from pyridine, pyrimidine, pyrazine, triazine and pyridazine rings;

p is an integer between 0 and 3 inclusive;

m is an integer between 0 and 5 inclusive;

p+m is an integer between 0 and 5;

the radicals R₁₁, which are identical or different, are a halogen atom; a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a carboxyl radical; a C₁-C₆ alkylcarbonyl radical; a thio radical; a C₁-C₆ thioalkyl radical; an alkyl(C₁-C₆)thio radical; an amino radical; an amino radical substituted by an alkyl(C₁-C₆), alkyl(C₁-C₆)carbonyl, amido or alkyl(C₁-C₆)sulphonyl radical; a C₁-C₆ monohydroxyalkyl radical; or a C₂-C₆ polyhydroxyalkyl radical, it being understood that the radicals R₁₁ are carried by a carbon atom;

the radicals R₁₂, which are identical or different, are a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; a C₁-C₆ carbamylalkyl radical; a C₁-C₆ alkyl(C₁-C₆)carboxyalkyl radical; or a benzyl radical, it being understood that the radicals R₁₂ are carried by a nitrogen;

R₁₃ is a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carboxyalkyl radical; a C₁-C₆ carbamylalkyl radical; a C₁-C₆ trifluoroalkyl radical; a C₁-C₆ trialkyl(C₁-C₆)silanalkyl radical; a C₁-C₆ sulphonamidoalkyl radical; a C₁-C₆ alkyl(C₁-C₆)carboxyalkyl radical; a C₁-C₆ alkyl(C₁-C₆)sulphinylalkyl radical; a C₁-C₆ alkyl(C₁-C₆)sulphonylalkyl radical; a C₁-C₆ alkyl(C₁-C₆)carbonylalkyl radical; a C₁-C₆ N-alkyl(C₁-C₆)carbamylalkyl radical; or a C₁-C₆ N-alkyl(C₁-C₆)sulphonamidoalkyl radical;

x is 0 or 1:

if x=0, the linking arm D is attached to the nitrogen atom;

if x=1, the linking arm D is attached to one of the vertices E, G, J, L or M; and

Y is a counterion.

Preferably, the vertices E, G, J, L and M form a pyridine and pyrimidine ring with the nitrogen in the ring.

If x is equal to 0, R₁₁ is preferably selected from a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a C₁-C₆ alkylcarbonyl radical; an amino radical; an amino radical monosubstituted or disubstituted by an alkyl(C₁-C₆), alkyl(C₁-C₆)carbonyl, amido or alkyl(C₁-C₆)sulphonyl radical; a C₁-C₆ monohydroxyalkyl radical; and a C₂-C₆ polyhydroxyalkyl radical; and R₁₂ is selected from a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; and a C₁-C₆ carbamylalkyl radical.

If x is equal to 1, R₁₃ is preferably selected from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carbonylalkyl radical; and a C₁-C₆ N-alkyl(C₁-C₆)carbamylalkyl radical; R₁₁ is selected from a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a C₁-C₆ alkylcarbonyl radical; an amino radical; and an amino radical monosubstituted or disubstituted by an alkyl(C₁-C₆), alkyl(C₁-C₆)carbonyl, amido or alkyl(C₁-C₆)sulphonyl radical; and R₁₂ is selected from a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; and a C₁-C₆ carbamylalkyl radical.

Preferably, R₁₁, R₁₂ and R₁₃ are alkyl radicals capable of being substituted.

The radical R₂ can also be an onium radical of the formula

—XP(O)(O—)OCH₂CH₂N⁺(CH₃)₃

where X is an oxygen atom or a radical —NR₁₄, R₁₄ being a hydrogen, a C₁-C₄ alkyl radical or a hydroxyalkyl radical.

Within the framework of the invention, R₂ can also be a guanidine radical of formula —X—C═NR₈—NR₉R₁₀, where X is an oxygen atom or a radical —NR₁₁, and R₈, R₉, R₁₀ and R₁₁ being a hydrogen, a C₁-C₄ alkyl radical or a hydroxyalkyl radical. In one particular embodiment, X is —NR₁₁, R₈ is a hydrogen and R₉ and R₁₀ are selected from hydrogen and an alkyl radical, preferably a methyl radical.

The pKa of the guanidine radical R₂ is generally such that this substituent is present in cationic form (═NR₈H⁺) under the conventional conditions of hair dyeing by oxidation.

Within the framework of the invention, the counterion can be derived from a halogen atom such as bromine, chlorine, fluorine or iodine, a hydroxide, a citrate, a succinate, a tartrate, a lactate, a tosylate, a mesylate, a benzenesulphonate, an acetate, a hydrogensulphate or a C₁-C₆ alkylsulphate, for example methylsulphate or ethylsulphate.

Within the framework of the present application, it is preferable to use the cationic tertiary paraphenylenediamines described above, containing a pyrrolidine ring, in which R₂ has formula II or III. It is even more preferable to use the cationic tertiary paraphenylenediamines described above, containing a pyrrolidine ring, in which R₂ has formula II or formula III where x=0, and in which n=0.

The following may be mentioned as examples of derivatives of formula (I):

| Formula | Nomenclature |
|---|---|
| 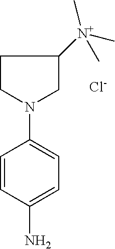 | [1-(4-Amino- phenyl)pyr- rolidin-3-yl-tri- methyl-ammonium chloride (1) |
| 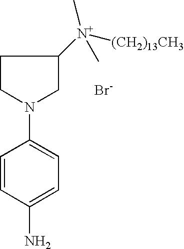 | [1-(4-Amino- phenyl)pyr- rolidin-3-yl]di- methyltetra- decyl-ammonium bromide (2) |
| 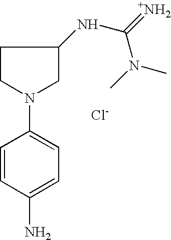 | N′-[1-(4-Amino- phenyl)pyr- rolidin-3-yl]-N,N-di- methyl guanidinium chloride (3) |
| 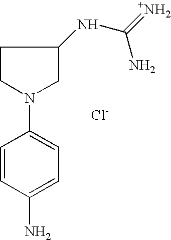 | N-[1-(4-Amino- phenyl)-pyr- rolidin-3-yl]guanidinium chloride (4) |
| 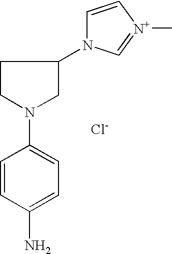 | 3-[1-(4-Amino- phenyl)pyr- rolidin-3-yl]-1-meth- yl-3H-imidazole-1-ium chloride (5) |
| 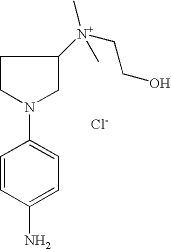 | [1-(4-Amino- phenyl)pyr- rolidin-3-yl]-(2-hy- droxy-ethyl)dimethyl- ammonium chloride (6) |
| 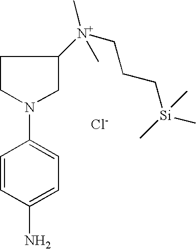 | [1-(4-Amino- phenyl)pyr- rolidin-3-yl]di- methyl-(3-tri- methylsilanylpro- pyl)ammonium chloride (7) |
| 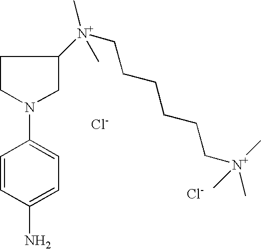 | [1-(4-Amino- phenyl)pyr- rolidin-3-yl]-(tri- methyl-ammonium- hexyl)dimethyl- ammonium dichloride (8) |
| 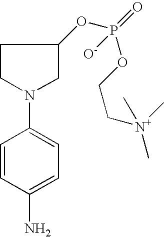 | [1-(4-Amino- phenyl)-pyr- rolidin-3-yl]oxo- phosphoryl choline (9) |
| 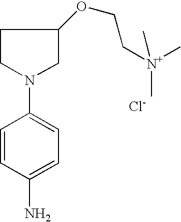 | {2-[1-(4-Amino- phenyl)-pyr- rolidin-3-yloxy]-eth- yl}-trimethyl-ammonium chloride (10) |
| 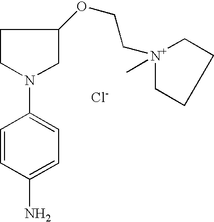 | 1-{2-[1-(4-Amino- phenyl)pyr- rolidin-3-yloxy]eth- yl}-1-methyl-pyr- rolidinium; chloride (11) |
| 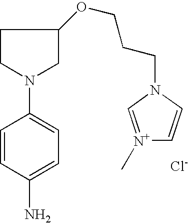 | 3-{3-[1-(4-Amino- phenyl)pyr- rolidin-3-yloxy]-pro- pyl}-1-meth- yl-3H-imidazol-1-ium; chloride (12) |
| 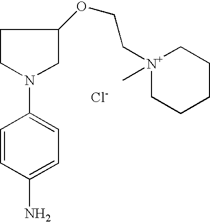 | 1-{2-[1-(4-Amino- phenyl)pyr- rolidin-3-yloxy]eth- yl}-1-methyl piperidinium; chloride (13) |
| 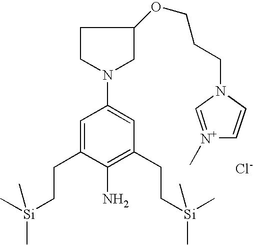 | 3-{3-[1-(5-tri- methylsilanyl- ethyl-4-Amino-3-tri- methylsilanyl-eth- ylphenyl)-pyr- rolidin-3-yloxy]pro- pyl}-1-meth- yl-3H-imidazole-1-um; chloride (14) |
| 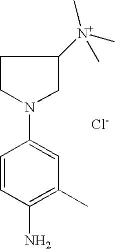 | [1-(4-Amino-3-meth- ylphenyl)pyrrolidin-3-yl]-tri- methylammonium- chloride (15) |
| 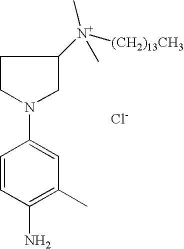 | [1-(4-Amino-3-meth- ylphenyl)-pyr- rolidin-3-yl]dimethyl- tetradecyl- ammonium chloride (16) |
| 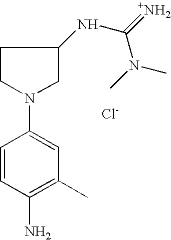 | N′-[1-(4-Amino-3-meth- ylphenyl)-pyr- rolidin-3-yl]-N,N-dimethyl guanidinium chloride (17) |
| 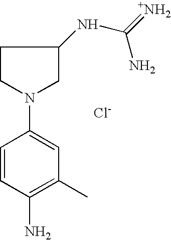 | N-[1-(4-Amino-3-meth- ylphenyl)-pyr- rolidin-3-yl]-guanidinium chloride (18) |
| 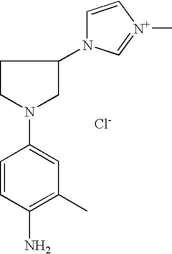 | 3-[1-(4-Amino-3-meth- ylphenyl)-pyr- rolidin-3-yl]-1-meth- yl-3H-imidazole-1-ium chloride (19) |
| 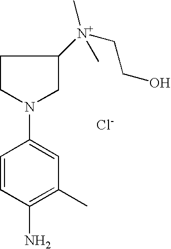 | [1-(4-Amino-3-meth- ylphenyl)-pyr- rolidin-3-yl]-(2-hy- droxyethyl)-dimethyl- ammonium chloride (20) |
| 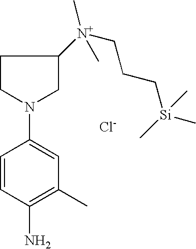 | [1-(4-Amino-3-meth- ylphenyl)pyr- rolidin-3-yl]-di- methyl(3-tri- methylsilanyl-pro- pylammonium chloride (21) |
| 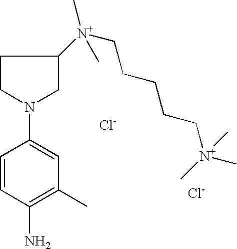 | [1-(4-Amino-3-meth- ylphenyl)-pyr- rolidin-3-yl]-(tri- methyl-ammonium-hex- yl)dimethyl-ammonium dichloride (22) |
| 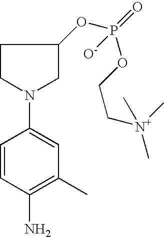 | [1-(4-Amino-3-meth- ylphenyl)-pyr- rolidin-3-yl]-oxo- phosphorylcholine (23) |
| 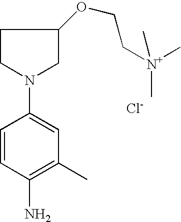 | {2-[1-(4-Amino-3-meth- ylphenyl)-pyr- rolidin-3-yloxy]eth- yl}-trimethyl-ammonium chloride (24) |
| 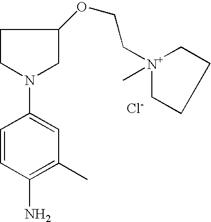 | 1-{2-[1-(4-Amino-3-meth- ylphenyl)pyr- rolidin-3-yloxy]eth- yl}-1-meth- yl-pyrrolidinium chloride (25) |
| 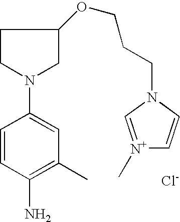 | 3-{3-[1-(4-Amino-3-meth- ylphenyl)-pyr- rolidin-3-yloxy]pro- pyl}-1-methyl-3H-imi- dazole-1-um chloride (26) |
| 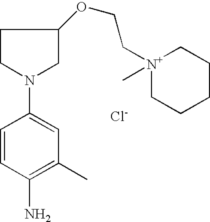 | 1-{2-[1-(4-Amino-3-meth- ylphenyl)pyr- rolidin-3-yloxy]eth- yl}-1-methyl- piperidinium chloride (27) |
| 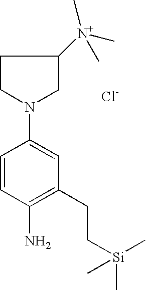 | [1-(4-Amino-3-tri- methylsilanylethylphe- nyl)-pyrrolidin-3-yl]-tri- methyl-ammonium chloride (28) |
| 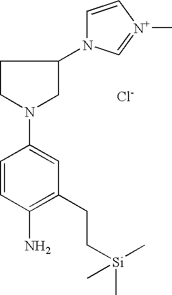 | 3-[1-(4-Amino-3-tri- methylsilanylethyl- phenyl)pyrroli- din-3-yl]-1-meth- yl-3H-imidazole-1-ium chloride (29) |
| 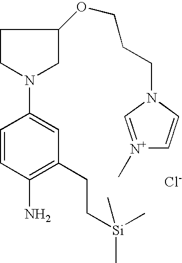 | 3-{3-[1-(4-Amino-3-tri- methylsilanylethyl- phenyl)-pyrroli- din-3-yloxy]pro- pyl}-1-methyl-3H-imi- dazole-1-um chloride (30) |
| 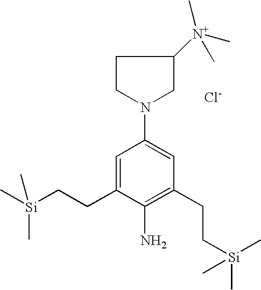 | [1-(5-trimethylsilanyleth- yl-4-Amino-3-tri- methylsilanyl- ethylphenyl)pyr- rolidin-3-yl]-tri- methylammonium- chloride (31) |
| 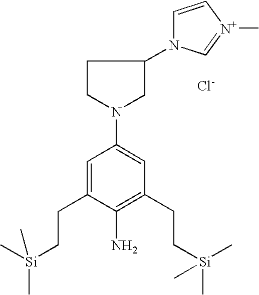 | 3-[1-(5-tri- methylsilanyleth- yl-4-Amino-3-tri- methylsilanyleth- ylphenyl)-pyr- rolidin-3-yl]-1-meth- yl-3H-imidazole-1-ium chloride (32) |
| 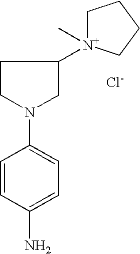 | 1′-(4-Amino- phenyl)-1-meth- yl-[1,3′]bi- pyrrolidinyl-1-ium chloride (33) |
| 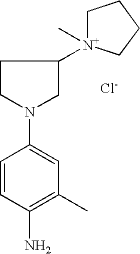 | 1′-(4-Amino-3-meth- ylphenyl)-1-meth- yl-[1,3′]bi- pyrrolidinyl-1-ium chloride (34) |
| 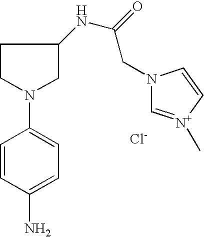 | 3-{[1-(4-Amino- phenyl)pyr- rolidin-3-ylcarba- moyl]-methyl}-1-meth- yl-3H-imidazole-1-ium chloride (35) |
| 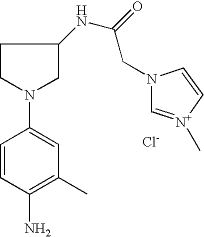 | 3-{[1-(4-Amino-3-methyl- phenyl)-pyr- rolidin-3-ylcarba- moyl]-meth- yl}-1-meth- yl-3H-imi- dazole-1-ium chloride (36) |
| 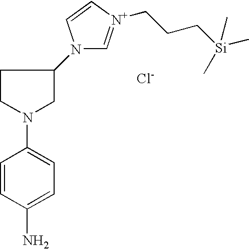 | 3-[1-(4-Amino- phenyl)pyr- rolidin-3-yl]-1-(3-tri- methylsilanyl- propyl)-3H-imi- dazole-1-ium chloride (37) |
| 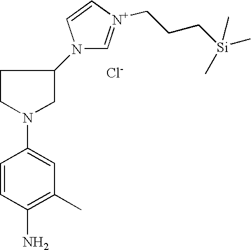 | 3-[1-(4-Amino- phenyl)pyr- rolidin-3-yl]-1-(3-tri- methylsilanyl-pro- pyl)-3H-imi- dazole-1-ium chloride (38) |
| 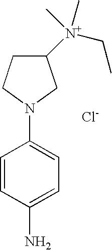 | [1-(4-Amino- phenyl)pyr- rolidin-3-yl]-eth- yldimethyl ammonium chloride (39) |
| 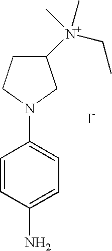 | [1-(4-Aminophenyl)pyr- rolidin-3-yl]-ethyl- dimethyl-ammonium iodide (40) |
| 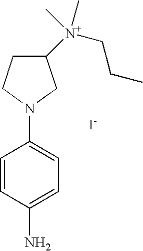 | [1-(4-amino- phenyl)pyr- rolidin-3-yl]-pro- pyldimethyl- ammonium iodide, (41) |
| 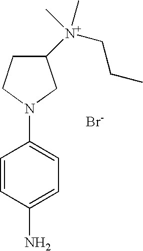 | [1-(4-Amino- phenyl)pyr- rolidin-3-yl]-pro- pyldimethyl-ammonium bromide (42) |
| 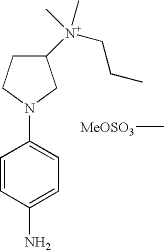 | [1-(4-Amino- phenyl)pyr- rolidin-3-yl]-pro- pyldimethyl- ammonium methosulphate (43) |
| 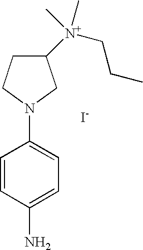 | [1-(4-Amino- phenyl)pyr- rolidin-3-yl]-butyl- dimethyl-ammonium iodide (44) |
| 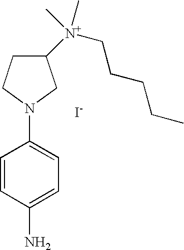 | [1-(4-Amino- phenyl)pyr- rolidin-3-yl]-pentyl- dimethylammonium iodide (45) |
| 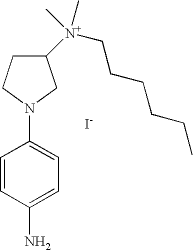 | [1-(4-Aminophenyl)pyr- rolidin-3-yl]-hexyl- dimethyl-ammonium iodide (46) |
| 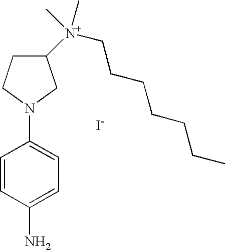 | [1-(4-Amino- phenyl)pyr- rolidin-3-yl]-heptyl- dimethyl-ammonium iodide (47) |
| 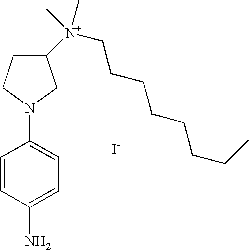 | [1-(4-Amino- phenyl)pyr- rolidin-3-yl]-octyl- dimethyl-ammonium iodide (48) |
| 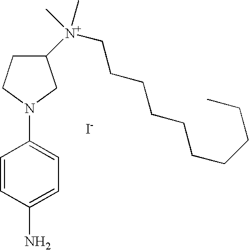 | [1-(4-Amino- phenyl)pyr- rolidin-3-yl]-decyl- dimethyl ammonium iodide (49) |
| 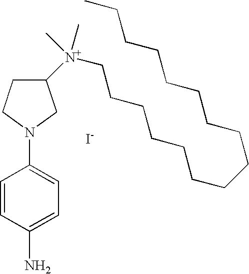 | [1-(4-amino-phenyl)pyr- rolidin-3-yl]-hexa- decyldimethylammonium iodide (50) |
| 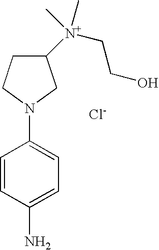 | [1-(4-Amino- phenyl)pyr- rolidin-3-yl]-hy- droxyethyl-dimethyl- ammonium chloride (51) |
| 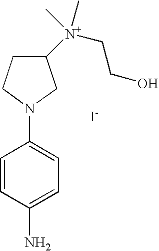 | [1-(4-amino- phenyl)-pyr- rolidin-3-yl]-hy- droxyethyl-dimethyl-am- monium iodide (52) |

Preferably, the following derivatives of formula I are used:

[1-(4-aminophenyl)pyrrolidin-3-yl]trimethylammonium; chloride;

[1-(4-aminophenyl)pyrrolidin-3-yl]dimethyltetradecylammonium; bromide;

N′-[1-(4-aminophenyl)pyrrolidin-3-yl]-N,N-dimethyl guanidinium chloride;

N-[1-(4-aminophenyl)pyrrolidin-3-yl] guanidinium chloride;

3-[1-(4-aminophenyl)pyrrolidin-3-yl]-1-methyl-3H-imidazol-1-ium; chloride;

[1-(4-aminophenyl)pyrrolidin-3-yl] (2-hydroxyethyl)dimethylammonium; chloride;

[1-(4-aminophenyl)pyrrolidin-3-yl]dimethyl(3-trimethylsilanylpropyl)ammonium; chloride;

[1-(4-amino-3-methylphenyl)pyrrolidin-3-yl]trimethylammonium; chloride;

[1-(4-amino-3-methylphenyl)pyrrolidin-3-yl]dimethyltetradecylammonium; chloride;

N′-[1-(4-amino-3-methylphenyl)pyrrolidin-3-yl]-N,N-dimethyl guanidinium chloride;

N-[1-(4-amino-3-methylphenyl)pyrrolidin-3-yl] guanidinium chloride;

3-[1-(4-amino-3-methylphenyl)pyrrolidin-3-yl]-1-methyl-3H-imidazol-1-ium; chloride;

[1-(4-amino-3-methylphenyl)pyrrolidin-3-yl] (2-hydroxyethyl)dimethylammonium; chloride;

[1-(4-amino-3-methylphenyl)pyrrolidin-3-yl]dimethyl(3-trimethylsilanylpropyl)ammonium; chloride;

1′-(4-aminophenyl)-1-methyl[1,3′]bipyrrolidinyl-1-ium; chloride;

1′-(4-amino-3-methylphenyl)-1-methyl[1,3′]bipyrrolidinyl-1-ium; chloride;

3-{[1-(4-aminophenyl)pyrrolidin-3-ylcarbamoyl]methyl}-1-methyl-3H-imidazol-1-ium; chloride;

3-{[1-(4-amino-3-methylphenyl)pyrrolidin-3-ylcarbamoyl]methyl}-1-methyl-3H-imidazol-1-ium; chloride;

3-[1-(4-aminophenyl)pyrrolidin-3-yl]-1-(3-trimethylsilanylpropyl)-3H-imidazol-1-ium; chloride;

3-[1-(4-amino-3-methylphenyl)pyrrolidin-3-yl]-1-(3-trimethylsilanyl-propyl)-3H-imidazol-1-ium; chloride;

[1-(4-aminophenyl)pyrrolidin-3-yl]ethyldimethylammonium; chloride;

[1-(4-aminophenyl)pyrrolidin-3-yl]ethyldimethylammonium; iodide;

[1-(4-aminophenyl)pyrrolidin-3-yl]propyldimethylammonium; iodide;

[1-(4-aminophenyl)pyrrolidin-3-yl]propyldimethylammonium; bromide;

[1-(4-aminophenyl)pyrrolidin-3-yl]propyldimethylammonium; methosulphate;

[1-(4-aminophenyl)pyrrolidin-3-yl]butyldimethylammonium; iodide;

[1-(4-aminophenyl)pyrrolidin-3-yl]pentyldimethylammonium; iodide;

[1-(4-aminophenyl)pyrrolidin-3-yl]hexyldimethylammonium; iodide;

[1-(4-aminophenyl)pyrrolidin-3-yl]heptyldimethylammonium; iodide;

[1-(4-aminophenyl)pyrrolidin-3-yl]octyldimethylammonium; iodide;

[1-(4-aminophenyl)pyrrolidin-3-yl]decyldimethylammonium; iodide;

[1-(4-aminophenyl)pyrrolidin-3-yl]hexadecyldimethylammonium; iodide;

[1-(4-aminophenyl)pyrrolidin-3-yl]hydroxyethyldimethylammonium; chloride; and

[1-(4-aminophenyl)pyrrolidin-3-yl]hydroxyethyldimethylammonium; iodide.

More preferably, the following compounds will be used:

[1-(4-aminophenyl)pyrrolidin-3-yl]trimethylammonium; chloride;

[1-(4-aminophenyl)pyrrolidin-3-yl]dimethyltetradecylammonium; bromide;

N′-[1-(4-aminophenyl)pyrrolidin-3-yl]-N,N-dimethyl guanidinium chloride;

N-[1-(4-aminophenyl)pyrrolidin-3-yl] guanidinium chloride;

3-[1-(4-aminophenyl)pyrrolidin-3-yl]-1-methyl-3H-imidazol-1-ium; chloride;

[1-(4-aminophenyl)pyrrolidin-3-yl](2-hydroxyethyl)dimethylammonium; chloride;

[1-(4-aminophenyl)pyrrolidin-3-yl]dimethyl(3-trimethylsilanylpropyl)ammonium; chloride;

[1-(4-aminophenyl)pyrrolidin-3-yl](trimethylammoniohexyl)dimethylammonium; dichloride;

1′-(4-aminophenyl)-1-methyl[1,3′]bipyrrolidinyl-1-ium; chloride;

3-[1-(4-aminophenyl)pyrrolidin-3-yl]-1-(3-trimethylsilanylpropyl)-3H-imidazol-1-ium; chloride;

3-[1-(4-amino-3-methylphenyl)pyrrolidin-3-yl]-1-(3-trimethylsilanyl-propyl)-3H-imidazol-1-ium; chloride;

[1-(4-aminophenyl)pyrrolidin-3-yl]ethyldimethylammonium; chloride;

[1-(4-aminophenyl)pyrrolidin-3-yl]ethyldimethylammonium; iodide;

[1-(4-aminophenyl)pyrrolidin-3-yl]propyldimethylammonium; iodide;

[1-(4-aminophenyl)pyrrolidin-3-yl]propyldimethylammonium; bromide;

[1-(4-aminophenyl)pyrrolidin-3-yl]propyldimethylammonium; methosulphate;

[1-(4-aminophenyl)pyrrolidin-3-yl]butyldimethylammonium; iodide;

[1-(4-aminophenyl)pyrrolidin-3-yl]pentyldimethylammonium; iodide;

[1-(4-aminophenyl)pyrrolidin-3-yl]hexyldimethylammonium; iodide;

[1-(4-aminophenyl)pyrrolidin-3-yl]heptyldimethylammonium; iodide;

[1-(4-aminophenyl)pyrrolidin-3-yl]octyldimethylammonium; iodide;

[1-(4-aminophenyl)pyrrolidin-3-yl]decyldimethylammonium; iodide;

[1-(4-aminophenyl)pyrrolidin-3-yl]hexadecyldimethylammonium; iodide;

[1-(4-aminophenyl)pyrrolidin-3-yl]hydroxyethyldimethylammonium; chloride; and

[1-(4-aminophenyl)pyrrolidin-3-yl]hydroxyethyldimethylammonium; iodide.

Even more preferably, the following compounds will be used:

[1-(4-aminophenyl)pyrrolidin-3-yl]trimethylammonium; chloride;

3-[1-(4-aminophenyl)pyrrolidin-3-yl]-1-methyl-3H-imidazol-1-ium; chloride;

[1-(4-aminophenyl)pyrrolidin-3-yl](2-hydroxyethyl)dimethylammonium; chloride; and

1′-(4-aminophenyl)-1-methyl[1,3′]bipyrrolidinyl-1-ium; chloride; and very particularly:

[1-(4-aminophenyl)pyrrolidin-3-yl]trimethylammonium; chloride; and

[1-(4-aminophenyl)pyrrolidin-3-yl](2-hydroxyethyl)dimethylammonium; chloride.

The counterion is not critical in terms of the result of the invention; any compound similar to the preferred compounds described above, but with a different counterion, forms an integral part of the preferred compounds.

The cationic tertiary paraphenylenediamine(s) containing a pyrrolidine ring represent from 0.001% to 10% and preferably from 0.005% to 6% by weight, based on the total weight of the composition.

The compounds of formula (I) can be synthesized by known methods, especially those described in patent application WO 02/45675.

The monosaccharides and disaccharides are carbohydrates of general formula (V):

Cₘ(H₂O)ₙ (V)

in which m denotes an integer between 3 and 16,

and n denotes an integer between 3 and 15.

The monosaccharides are carbohydrates containing from 3 to 8 carbon atoms; these are aldehydes or ketones possessing from 2 to 7 hydroxyl groups, depending on the number of carbon atoms.

The monosaccharides containing an aldehyde group are conventionally called “aldoses” and the monosaccharides containing a ketone group are conventionally called “ketoses”.

The compositions according to the invention comprise at least one monosaccharide and at least one disaccharide or a mixture of monosaccharides and disaccharides.

The monosaccharides which can preferably be used in the compositions according to the present application contain 4, 5, 6 or 7 carbon atoms; these are tetroses, pentoses, hexoses and heptoses, respectively.

In view of the presence of asymmetric carbons—one for the trioses (monosaccharides containing 3 carbon atoms) and several for the other monosaccharides—each monosaccharide takes the form of several stereoisomers, which can be enantiomers in the case of the trioses and enantiomers and diastereoisomers in the case of the other monosaccharides.

In addition, the pentoses and hexoses which can be used within the framework of the present application can cyclize to furanose and pyranose rings, respectively; these furanose and pyranose rings are also monosaccharides which can be used within the framework of the present application.

Preferably, the aldoses which can be used within the framework of the present application are selected from the following monosaccharides: erythrose and threose, which are tetroses, ribose, arabinose, xylose and lyxose, which are pentoses, and allose, altrose, glucose, mannose, gulose, idose, galactose and talose, which are hexoses.

In particular, glucose has two cyclic forms: α-D-glucopyranose and β-D-glucopyranose; these two forms of glucose are monosaccharides which can be used within the framework of the present application.

Preferably, the ketoses which can be used within the framework of the present application are selected from the following monosaccharides: erythrulose, which is a tetrose, ribulose and xylulose, which are pentoses, and psicose, fructose, sorbose and tagatose, which are hexoses.

The ketoses can also have cyclic forms. By way of example, fructose can have the fructofuranose or fructopyranose form.

The disaccharides are carbohydrates formed of two identical or different molecules of monosaccharides bonded by a glycosidic O linkage. Preferably, the disaccharides which can be used within the framework of the present application contain 10, 11 or 12 carbon atoms.

Even more preferably, the disaccharides which can be used within the framework of the present application are selected from sucrose (α-D-glucopyranosyl(1→2)-β-D-fructofuranoside), lactose (β-D-galactopyranosyl(1→4)-α-D-glucopyranose) and maltose (α-D-glucopyranosyl(1→4)-α-D-glucopyranose).

The monosaccharide(s) and/or disaccharide(s) which can be used within the framework of the present application represent from 0.01 to 20% and preferably from 0.05 to 5% by weight, based on the total weight of the composition.

In a first preferred embodiment, the composition according to the present invention also contains at least one cationic polymer. In terms of the present invention, the expression “cationic polymer” denotes any polymer containing cationic groups and/or groups ionizable to cationic groups.

The cationic polymers which can be used according to the present invention can be selected from all those that are already known per se to improve the cosmetic properties of hair, namely, in particular, those described in patent application EP-A-337 354 and in French patents FR-2 270 846, 2 383 660, 2 598 611, 2 470 596 and 2 519 863.

The preferred cationic polymers are selected from those which comprise units containing primary, secondary, tertiary and/or quaternary amine groups capable either of forming part of the main polymer chain or of being carried by a lateral substituent directly joined to said chain.

The cationic polymers used generally have a number-average molecular weight of between about 500 and 5. 106 and preferably of between about 103 and 3.106.

Polymers of the polyamine, polyaminoamide and poly(quaternary ammonium) type may be mentioned more particularly among the cationic polymers.

These are known products. They are described especially in French patent no. 2 505 348 or 2 542 997. The following may be mentioned among said polymers:

(1) Homopolymers or copolymers derived from acrylic or methacrylic esters or amides and containing at least one of the units of formulae (VI), (VII), (VIII) and (IX) below:

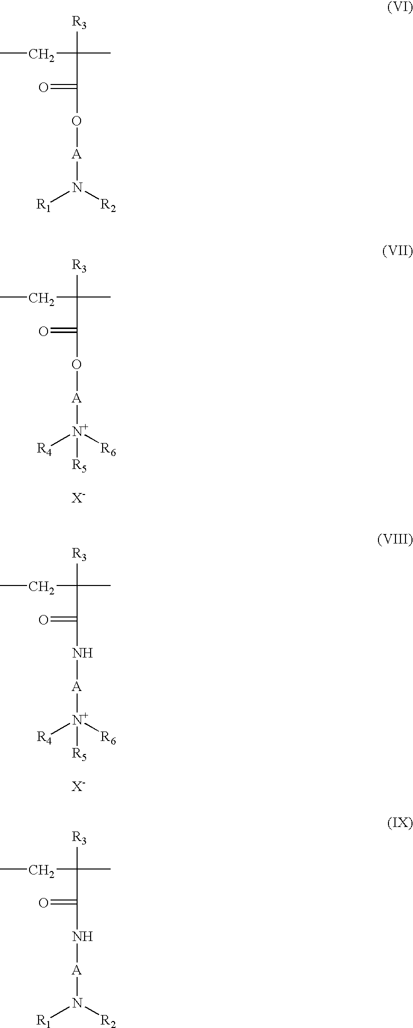

in which:

R₃ is a hydrogen atom or a CH₃ radical;

A is a linear or branched alkyl group containing from 1 to 6 carbon atoms and preferably 2 or 3 carbon atoms, or a hydroxyalkyl group containing from 1 to 4 carbon atoms;

R₄, R₅ and R₆, which are identical or different, are an alkyl group having from 1 to 18 carbon atoms or a benzyl radical, preferably an alkyl group having from 1 to 6 carbon atoms;

R₁ and R₂, which are identical or different, are hydrogen or an alkyl group having from 1 to 6 carbon atoms, preferably methyl or ethyl; and

X⁻ is an anion derived from a mineral or organic acid, such as a methosulphate anion or a halide like chloride or bromide.

The polymers of family (1) can also contain one or more units derived from comonomers which can be selected from the family comprising acrylamides, methacrylamides, diacetone acrylamides, acrylamides and methacrylamides substituted on the nitrogen by lower alkyls (C₁-C₄), acrylic or methacrylic acids or their esters, vinyllactams such as vinylpyrrolidone or vinylcaprolactam, and vinyl esters.

The following may thus be mentioned among these polymers of family (1):

acrylamide/dimethylaminoethyl methacrylate copolymers quaternized with dimethyl sulphate or a dimethyl halide, such as that sold under the name HERCOFLOC by HERCULES;

the acrylamide/methacryloyloxyethyltrimethylammonium chloride copolymers described e.g. in patent application EP-A-080 976 and sold under the name BINA QUAT P 100 by CIBA GEIGY;

the acrylamide/methacryloyloxyethyltrimethylammonium methosulphate copolymer sold under the name RETEN by HERCULES;

quaternized or non-quaternized vinylpyrrolidone/dialkylaminoalkyl acrylate or methacrylate copolymers such as the products sold under the name “GAFQUAT” by ISP, for example “GAFQUAT 734” or “GAFQUAT 755”, or the products known as “COPOLYMER 845, 958 and 937”; these polymers are described in detail in French patents 2.077.143 and 2.393.573;

dimethylaminoethyl methacrylate/vinylcaprolactam/vinylpyrrolidone terpolymers such as the product sold under the name GAFFIX VC 713 by ISP;

the vinylpyrrolidone/methacrylamidopropyldimethylamine copolymers marketed especially under the name STYLEZE CC 10 by ISP; and

quaternized vinylpyrrolidone/dimethylaminopropylmethacrylamide copolymers such as the product sold under the name “GAFQUAT HS 100” by ISP.

(2) The cellulose ether derivatives containing quaternary ammonium groups described in French patent 1 492 597, and particularly the polymers marketed under the name “JR” (JR 400, JR 125, JR 30M) or “LR” (LR 400, LR 30M) by Union Carbide Corporation. These polymers are also defined in the CTFA dictionary as quaternary ammonium compounds of hydroxyethyl cellulose which have reacted with an epoxide substituted by a trimethylammonium group.

(3) The cationic cellulose derivatives, such as the cellulose copolymers or the cellulose derivatives grafted with a water-soluble quaternary ammonium monomer, described especially in patent U.S. Pat. No. 4,131,576, such as hydroxyalkyl celluloses, like hydroxymethyl, hydroxyethyl or hydroxypropyl celluloses, grafted especially with a methacryloylethyltrimethylammonium, methacrylamidopropyltrimethylammonium or dimethyldiallylammonium salt.

The marketed products corresponding to this definition are more particularly the products sold under the name “Celquat L 200” and “Celquat H 100” by National Starch.

(4) The cationic polysaccharides described more particularly in U.S. Pat. Nos. 3,589,578 and 4 031 307, such as guar gums containing cationic trialkylammonium groups. For example, guar gums modified by a 2,3-epoxy-propyltrimethylammonium salt (e.g. chloride) are used.

Such products are marketed especially under the trade names JAGUAR C 13 S, JAGUAR C 15, JAGUAR C 17 or JAGUAR C 162 by MEYHALL.

(5) Polymers consisting of piperazinyl units and divalent alkylene or hydroxyalkylene radicals with linear or branched chains optionally interrupted by oxygen, sulphur or nitrogen atoms or by aromatic or heterocyclic rings, as well as the oxidation and/or quaternization products of these polymers. Such polymers are described especially in French patents 2.162.025 and 2.280.361.

(6) Water-soluble polyaminoamides prepared in particular by the polycondensation of an acidic compound with a polyanine; these polyaminoamides can be crosslinked with an epihalogenohydrin, a diepoxide, a dianhydride, an unsaturated dianhydride, a bis-unsaturated derivative, a bis-halogenohydrin, a bis-azetidinium compound, a bis-halogenoacyldiamine or a bis(alkyl halide) or with an oligomer resulting from the reaction of a reactive bifunctional compound with a bis-halogenohydrin, a bis-azetidinium compound, a bis-halogenoacyldiamine, a bis(alkyl halide), an epihalogenohydrin, a diepoxide or a bis-unsaturated derivative, the crosslinking agent being used in proportions ranging from 0.025 to 0.35 mol per amine group of the polyaminoamide; these polyaminoamides can be alkylated or, if they contain one or more tertiary amine groups, they can be quaternized. Such polymers are described especially in French patents 2.252.840 and 2.368.508.

(7) Polyaminoamide derivatives resulting from the condensation of polyalkylenepolyamines with polycarboxylic acids, followed by alkylation with bifunctional agents. Examples which may be mentioned are adipic acid/dialkylaminohydroxyalkyldialkylenetriamine polymers in which the alkyl radical contains from 1 to 4 carbon atoms and is preferably methyl, ethyl or propyl. Such polymers are described especially in French patent 1.583.363.

The adipic acid/dimethylaminohydroxypropyldiethylenetriamine polymers sold under the name “Cartaretine F, F4 or F8” by Sandoz may be mentioned more particularly among these derivatives.

(8) Polymers obtained by reacting a polyalkylenepolyamine containing two primary amine groups and at least one secondary amine group with a dicarboxylic acid selected from diglycolic acid and saturated aliphatic dicarboxylic acids having from 3 to 8 carbon atoms. The molar ratio of polyalkylenepolyamine to dicarboxylic acid is between 0.8:1 and 1.4:1, the resulting polyaminoamide being reacted with epichlorohydrin in a molar ratio of epichlorohydrin to polyaminoamide secondary amine group of between 0.5:1 and 1.8:1. Such polymers are described especially in U.S. Pat. Nos. 3,227,615 and 2.961.347.

Polymers of this type are marketed in particular under the name “Hercosett 57” by Hercules Inc. or under the name “PD 170” or “Delsette 101” by Hercules in the case of the adipic acid/epoxypropyldiethylenetriamine copolymer.

(9) Cyclic polymers of alkyldiallylamine or dialkyldiallylammonium, such as homopolymers or copolymers containing, as the main constituent of the chain, units of formula (X) or (XI):

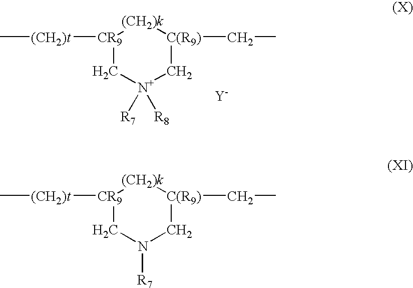

in which k and t are equal to 0 or 1, the sum k+t being equal to 1; R₉ is a hydrogen atom or a methyl radical; R₇ and R₈ independently of one another are an alkyl group having from 1 to 6 carbon atoms, a hydroxyalkyl group in which the alkyl group preferably has 1 to 5 carbon atoms, or a lower amidoalkyl group (C₁-C₄), or R₇ and R₈, together with the nitrogen atom to which they are attached, can be heterocyclic groups such as piperidinyl or morpholinyl, R₇ and R₈ independently of one another preferably being an alkyl group having from 1 to 4 carbon atoms; and Y is an anion such as bromide, chloride, acetate, borate, citrate, tartrate, bisulphate, bisulphite, sulphate or phosphate. These polymers are described especially in French patent 2.080.759 and in its certificate of addition 2.190.406.

Among the polymers defined above, the dimethyldiallylammonium chloride homopolymer sold under the name “Merquat 100” by Calgon (and its low weight-average molecular weight homologues) and the diallyldimethylammonium chloride/acrylamide copolymers marketed under the name “MERQUAT 550” may be mentioned more particularly.

(10) The quaternary diammonium polymer containing repeat units of the formula:

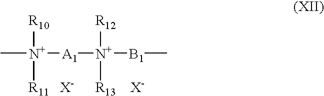

in which:

R₁₀, R₁₁, R₁₂ and R₁₃, which are identical or different, are aliphatic, alicyclic or arylaliphatic radicals containing from 1 to 20 carbon atoms or lower hydroxyalkylaliphatic radicals, or R₁₀, R₁₁, R₁₂ and R₁₃ together or separately form, with the nitrogen atoms to which they are attached, heterocycles optionally containing a second heteroatom other than nitrogen, or R₁₀, R₁₁, R₁₂ and R₁₃ are a linear or branched C₁-C₆ alkyl radical substituted by a nitrile, ester, acyl or amide group or a group —CO—O—R₁₄-D or —CO—NH—R₁₄-D, where R₁₄ is an alkylene and D is a quaternary ammonium group;

A₁ and B₁ are polymethylene groups containing from 2 to 20 carbon atoms which can be linear or branched and saturated or unsaturated and can contain, bonded to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups; and

X⁻ is an anion derived from a mineral or organic acid.

A₁, R₁₀ and R₁₂ can form a piperazine ring with the two nitrogen atoms to which they are attached; also, if A₁ is a saturated or unsaturated, linear or branched alkylene or hydroxyalkylene radical, B, can also be a group —(CH₂)ₙ—CO-D-OC—(CH₂)ₙ—, in which D is:

a) a glycol radical of formula —O-Z-O—, where Z is a linear or branched hydrocarbon radical or a group having one of the following formulae:

—(CH₂—CH₂—O)ₓ—CH₂—CH₂—

—[CH₂—CH(CH₃)—O]_{y}—CH₂—CH(CH₃)—

where x and y are integers from 1 to 4, representing a defined and unique degree of polymerization, or any number from 1 to 4, representing a mean degree of polymerization;

b) a bis-secondary diamine radical such as a piperazine derivative;

c) a bis-primary diamine radical of formula —NH—Y—NH—, where Y is a linear or branched hydrocarbon radical or the following bivalent radical:

—CH₂—CH₂—S—S—CH₂—CH₂—;

or

d) a ureylene group of formula —NH—CO—NH—.

Preferably, X⁻ is an anion such as chloride or bromide.

These polymers have a number-average molecular weight generally of between 1000 and 100,000.

Polymers of this type are described especially in French patents 2.320.330, 2.270.846, 2.316.271, 2.336.434 and 2.413.907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Polymers which can be used more particularly are those consisting of repeat units of formula (XIII) below:

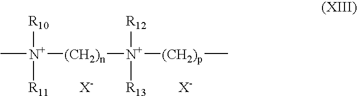

in which R₁₀, R₁₁, R₁₂ and R₁₃, which are identical or different, are an alkyl or hydroxyalkyl radical having from about 1 to 4 carbon atoms, n and p are integers varying from 2 to 20 and X is an anion derived from a mineral or organic acid.

(11) The poly(quaternary ammonium) polymers consisting of repeat units of formula (XIV):

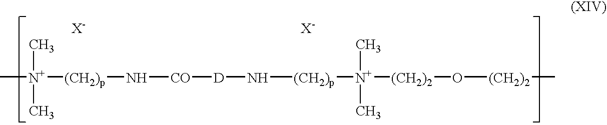

in which p is an integer varying from about 1 to 6, D can be nothing or a group —(CH₂)ᵣ—CO—, in which r is a number equal to 4 or 7, and X⁻ is an anion.

Such polymers can be prepared by the processes described in U.S. Pat. Nos. 4,157,388, 4,702,906 and 4,719,282. They are described especially in patent application EP-A-122 324.

The products “Mirapol A 15”, “Mirapol AD1”, “Mirapol AZ1” and “Mirapol 175”, sold by Miranol, may be mentioned as examples of these polymers.

(12) Quaternary polymers of vinylpyrrolidone and vinylimidazole, for example the products marketed under the names Luviquat FC 905, FC 550 and FC 370 by BASF.

(13) Polyamines such as Polyquart H sold by HENKEL, which is referred to by the name “POLYETHYLENE GLYCOL (15) TALLOW POLYAMINE” in the CTFA dictionary.

(14) Crosslinked polymers of methacryloyloxyalkyl(C₁-C₄)trialkyl(C₁-C₄)— ammonium salts, such as the polymers obtained by the homopolymerization of dimethylaminoethyl methacrylate quaternized by methyl chloride, or by the copolymerization of acrylamide with dimethylaminoethyl methacrylate quaternized by methyl chloride, the homopolymerization or copolymerization being followed by crosslinking with an olefinically unsaturated compound, particularly methylene-bis-acrylamide. More particularly, it is possible to use a crosslinked acrylamide/methacryloyloxyethyltrimethylammonium chloride copolymer (20/80 by weight) in the form of a dispersion containing 50% by weight of said copolymer in mineral oil. This dispersion is marketed under the name “SALCARE® SC 92” by ALLIED COLLOIDS. It is also possible to use a crosslinked methacryloxyethyltrimethylammonium chloride homopolymer containing about 50% by weight of the homopolymer in mineral oil or a liquid ester. These dispersions are marketed under the names “SALCARE® SC 95” and “SALCARE® SC 96” by ALLIED COLLOIDS.

Other cationic polymers which can be used within the framework of the invention are polyalkylenimines, particularly polyethylenimines, polymers containing vinylpyridine or vinylpyridinium units, condensation products of polyamines and epichlorohydrin, quaternary polyureylenes and chitin derivatives.

Of all the cationic polymers capable of being used within the framework of the present invention, it is preferable to use polymers of families (1), (9), (10), (11) and (14) and particularly preferable to use polymers with repeat units of formulae (W) and (U) below:

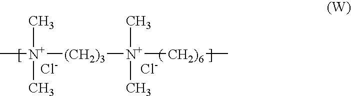

and especially those with a molecular weight of between 9500 and 9900, as determined by gel permeation chromatography;

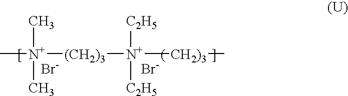

and especially those with a molecular weight of about 1200, as determined by gel permeation chromatography.

The concentration of cationic polymer in the composition according to the present invention can vary from 0.01 to 10% by weight, based on the total weight of the composition, preferably from 0.05 to 5% and particularly preferably from 0.1 to 3%.

In a second preferred embodiment, the composition according to the present invention also contains at least one thickening polymer, or “rheology adjuster”.

The rheology adjusters can be selected from fatty acid amides (copra diethanolamide or monoethanolamide, ethoxylated alkyl-ether-carboxylic acid monoethanolamide), cellulose thickeners (hydroxyethyl cellulose, hydroxypropyl cellulose, carboxymethyl cellulose), guar gum and its derivatives (hydroxypropyl guar), gums of microbial origin (xanthan gum, scleroglucan gum), crosslinked acrylic acid or acrylamidopropanesulphonic acid homopolymers, and associative polymers such as those described below.

The associative polymers which can be used according to the invention are water-soluble polymers that are capable of associating reversibly with one another or with other molecules in an aqueous medium.

Their chemical structure comprises hydrophilic zones and hydrophobic zones, the latter being characterized by at least one fatty chain.

The associative polymers which can be used according to the invention can be of anionic, cationic, amphoteric and, preferably, non-ionic type.

Their concentration by weight in the dyeing composition can vary from about 0.01 to 10% of the total weight of the composition, and their concentration by weight in the ready-to-use composition (including the oxidizing agent) can vary from about 0.0025 to 10% of the total weight of the composition. More preferably, this amount varies from about 0.1 to 5% by weight in the dyeing composition and from about 0.025 to 10% in the ready-to-use composition.

The following may be mentioned among the associative polymers of anionic type:

(I) those containing at least one hydrophilic unit and at least one allyl ether unit with a fatty chain, more particularly those whose hydrophilic unit consists of an ethylenically unsaturated anionic monomer, even more particularly of a vinyl-carboxylic acid and very particularly of an acrylic acid or a methacrylic acid, or mixtures thereof, and whose allyl ether unit with a fatty chain corresponds to the monomer of formula (XV) below:

CH₂═CR′CH₂OBₙR (XV)

in which R′ is H or CH₃, B is the ethylenoxy radical, n is zero or an integer ranging from 1 to 100 and R is a hydrocarbon radical selected from alkyl, arylalkyl, aryl, alkylaryl and cycloalkyl radicals containing from 8 to 30 carbon atoms, preferably 10 to 24 and more particularly from 12 to 18 carbon atoms. A more particularly preferred unit of formula (XV) is one in which R′ is H, n is equal to 10 and R is a stearyl radical (C₁₈).

Anionic associative polymers of this type are described and prepared by an emulsion polymerization process in patent EP-0 216 479.

Among these anionic associative polymers, those which are particularly preferred according to the invention are the polymers formed from 20 to 60% by weight of acrylic acid and/or methacrylic acid, 5 to 60% by weight of lower alkyl (meth)acrylates, 2 to 50% by weight of allyl ether with a fatty chain of formula (XV) and 0 to 1% by weight of a crosslinking agent which is a well-known copolymerizable, polyethylenically unsaturated monomer such as diallyl phthalate, allyl (meth)acrylate, divinylbenzene, (poly)ethylene glycol dimethacrylate or methylene-bis-acrylamide.

Among the latter, those which are very particularly preferred are crosslinked terpolymers of methacrylic acid, ethyl acrylate and stearyl alcohol polyethylene glycol (10 EO) ether (Steareth 10), especially those sold by ALLIED COLLOIDS under the names SALCARE SC800® and SALCARE SC90®, which are 30% aqueous emulsions of a crosslinked terpolymer of methacrylic acid, ethyl acrylate and steareth-10-allyl ether (40/50/10).

(II) those containing at least one hydrophilic unit of the olefinically unsaturated carboxylic acid type and at least one hydrophobic unit of the unsaturated carboxylic acid alkyl (C₁₀-C₃₀) ester type.

Preferably, these polymers are selected from those whose hydrophilic unit of the olefinically unsaturated carboxylic acid type corresponds to the monomer of formula (XVI) below:

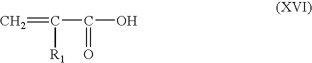

in which R₁ is H, CH₃ or C₂H₅, i.e. acrylic acid, methacrylic acid or ethacrylic acid units, and whose hydrophobic unit of the unsaturated carboxylic acid alkyl (C₁₀-C₃₀) ester type corresponds to the monomer of formula (XVII) below:

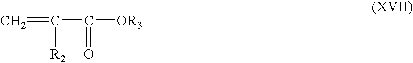

in which R₂ is H, CH₃ or C₂H₅ (i.e. acrylate, methacrylate or ethacrylate units) and preferably H (acrylate units) or CH₃ (methacrylate units), R₃ being a C₁₀-C₃₀ and preferably C₁₂-C₂₂ alkyl radical.

Unsaturated carboxylic acid alkyl (C₁₀-C₃₀) esters according to the invention include e.g. lauryl acrylate, stearyl acrylate, decyl acrylate, isodecyl acrylate, dodecyl acrylate and the corresponding methacrylates, namely lauryl methacrylate, stearyl methacrylate, decyl methacrylate, isodecyl methacrylate and dodecyl methacrylate.

Anionic polymers of this type are described and prepared e.g. in U.S. Pat. Nos. 3,915,921 and 4,509,949.

Polymers which will be used more particularly among the anionic associative polymers of this type are those formed from a mixture of monomers comprising:

(i) essentially acrylic acid;

(ii) an ester of formula (XVI) described above in which R₂ is H or CH₃, R₃ being an alkyl radical having from 12 to 22 carbon atoms; and

(iii) a crosslinking agent which is a well-known copolymerizable, poly-ethylenically unsaturated monomer such as diallyl phthalate, allyl (meth)acrylate, divinylbenzene, (poly)ethylene glycol dimethacrylate or methylene-bis-acrylamide.

The polymers which will be used more particularly among the anionic associative polymers of this type are those consisting of 95 to 60% by weight of acrylic acid (hydrophilic unit), 4 to 40% by weight of C₁₀-C₃₀ alkyl acrylate (hydrophobic unit) and 0 to 6% by weight of crosslinking polymerizable monomer, or those consisting of 98 to 96% by weight of acrylic acid (hydrophilic unit), 1 to 4% by weight of C₁₀-C₃₀ alkyl acrylate (hydrophobic unit) and 0.1 to 0.6% by weight of a crosslinking polymerizable monomer such as those described above.

Among said polymers mentioned above, those which are very particularly preferred according to the present invention are the products sold by GOODRICH under the trade names PEMULEN TR1®, PEMULEN TR2® and CARBOPOL 1382®, particularly preferably PEMULEN TR1, and the product sold by SEPPIC under the name COATEX SX®.

(III) maleic anhydride/C₃₀-C₃₈ α-olefin/alkyl maleate terpolymers such as the product (maleic anhydride/C₃₀-C₃₈ α-olefin/isopropyl maleate copolymer) sold under the name PERFORMA V 1608′ by NEWPHASE TECHNOLOGIES.

(IV) acrylic terpolymers comprising:

(a) about 20% to 70% by weight of an α,β-monoethylenically unsaturated carboxylic acid;

(b) about 20 to 80% by weight of a non-surface-active α,β-monoethylenically unsaturated monomer other than (a); and

(c) about 0.5 to 60% by weight of a non-ionic monourethane which is the reaction product of a monohydric surfactant with a monoethylenically unsaturated monoisocyanate,

such as those described in patent application EP-A-0 173 109 and more particularly the one described in Example 3, namely a methacrylic acid/methyl acrylate/ethoxylated (40 EO) behenyl alcohol dimethylmetaisopropenylbenzyl isocyanate terpolymer in 25% aqueous dispersion.

(V) copolymers whose monomers include an α,β-monoethylenically unsaturated carboxylic acid and an α,β-monoethylenically unsaturated carboxylic acid ester of an alkoxylated fatty alcohol.

Preferably, another monomer included in these compounds is an α,β-monoethylenically unsaturated carboxylic acid ester of a C₁-C₄ alcohol.

An example of this type of compound which may be mentioned is ACULYN 22® sold by ROHM and HAAS, which is a methacrylic acid/ethyl acrylate/alkoxylated stearyl methacrylate terpolymer.

The following may be mentioned among the associative polymers of cationic type:

(I) the cationic associative polyurethanes whose family has been described by the Applicant in French patent application no. 0 009 609; it can be represented by general formula (XVIII) below:

R—X—(P)ₙ-[L-(Y)ₘ]ᵣ-L′-(P′)ₚ—X′-R′ (XVII)

in which:

R and R′, which are identical or different, are a hydrophobic group or a hydrogen atom;

X and X′, which are identical or different, are a group containing an amine functional group that may or may not carry a hydrophobic group, or the group L″;

L, L′ and L″, which are identical or different, are a group derived from a diisocyanate;

P and P′, which are identical or different, are a group containing an amine group that may or may not carry a hydrophobic group;

Y is a hydrophilic group;

r is an integer between 1 and 100, preferably between 1 and 50 and particularly between 1 and 25; and

n, m and p each have values, independently of the others, of between 0 and 1000,

the molecule containing at least one protonated or quaternized amine group and at least one hydrophobic group.

In one preferred embodiment of these polyurethanes, the only hydrophobic groups are the groups R and R′ at the ends of the chain.

A preferred family of cationic associative polyurethanes is the one of formula (XVEII) described above in which:

R and R′ are both independently a hydrophobic group;

X and X′ are each a group L″;

n and p have values of between 1 and 1000; and

L, L′, L″,P, P′,Y and m are as defined above.

Another preferred family of cationic associative polyurethanes is the one of formula (XVIII) above in which:

R and R′ are both independently a hydrophobic group;

X and X′ are each a group L”;

n and p have the value 0; and

L, L′, L″, Y and m are as defined above.

The fact that n and p have the value 0 means that these polymers do not contain units derived from a monomer with an amine functional group, incorporated into the polymer during polycondensation. The protonated amine groups of these polyurethanes result from the hydrolysis of excess isocyanate groups at the end of the chain, followed by alkylation of the resulting primary amine functional groups with alkylating agents having a hydrophobic group, i.e. compounds of the type RQ or R′Q, in which R and R′ are as defined above and Q is a leaving group such as a halide, a sulphate, etc.

Yet another preferred family of cationic associative polyurethanes is the one of formula (Ia) above in which:

R and R′ are both independently a hydrophobic group;

X and X′ are both independently a group containing a quaternary amine;

n and p have the value zero; and

L, L′, Y and m are as defined above.

The number-average molecular weight of the cationic associative polyurethanes is preferably between 400 and 500,000, particularly between 1000 and 400,000 and ideally between 1000 and 300,000.

Hydrophobic group is understood as meaning a radical or polymer with a linear or branched, saturated or unsaturated hydrocarbon chain capable of containing one or more heteroatoms such as P, O, N or S, or a radical with a perfluorinated or siliconized chain. If it is a hydrocarbon radical, the hydrophobic group contains at least 10 carbon atoms, preferably from 10 to 30 carbon atoms, particularly from 12 to 30 carbon atoms and more preferably from 18 to 30 carbon atoms.

Preferably, the hydrocarbon group is derived from a monofunctional compound.

By way of example, the hydrophobic group can be derived from a fatty alcohol such as stearyl alcohol, dodecyl alcohol or decyl alcohol. It can also be a hydrocarbon polymer, for example polybutadiene.

If X and/or X′ are a group containing a tertiary or quaternary amine, X and/or X′ can have one of the following formulae:

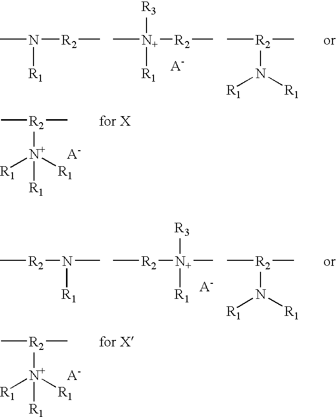

in which:

R₂ is a linear or branched alkylene radical having from 1 to 20 carbon atoms which may or may not contain a saturated or unsaturated ring, or an arylene radical, it being possible for one or more of the carbon atoms to be replaced by a heteroatom selected from N, S, O and P;

R₁ and R₃, which are identical or different, are a linear or branched C₁-C₃₀ alkyl or alkenyl radical, or an aryl radical, it being possible for at least one of the carbon atoms to be replaced by a heteroatom selected from N, S, O and P; and

A⁻0 is a physiologically acceptable counterion.

The groups L, L′ and L″ are a group of the formula:

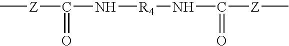

in which:

Z is —O—, —S— or —NH—; and

R₄ is a linear or branched alkylene radical having from 1 to 20 carbon atoms which may or may not contain a saturated or unsaturated ring, or an arylene radical, it being possible for one or more of the carbon atoms to be replaced by a heteroatom selected from N, S, O and P.

The groups P and P′ comprising an amine functional group can have at least one of the following formulae:

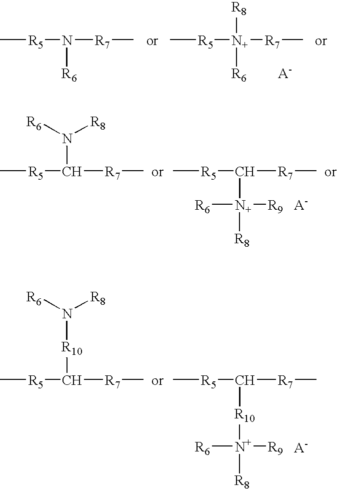

in which:

R₅ and R₇ are as defined above for R₂;

R₆, R₈ and R₉ are as defined above for R₁ and R₃;

R₁₀ is an optionally unsaturated, linear or branched alkylene group capable of containing one or more heteroatoms selected from N, O, S and P; and

A⁻ is a physiologically acceptable counterion.

As far as the meaning of Y is concerned, hydrophilic group is understood as meaning a polymeric or non-polymeric water-soluble group.

Examples which may be mentioned when polymers are not involved are ethylene glycol, diethylene glycol and propylene glycol.

If, according to one preferred embodiment, a hydrophilic polymer is involved, examples which may be mentioned are polyethers, sulphonated polyesters, sulphonated polyamides or a mixture of these polymers. Preferably, the hydrophilic compound is a polyether, especially a poly(ethylene oxide) or poly(propylene oxide).

The cationic associative polyurethanes of formula (XVIII) which can be used according to the invention are formed from diisocyanates and different compounds possessing groups with a labile hydrogen. The groups with a labile hydrogen can be alcohol, primary or secondary amine or thiol groups, which, after reaction with the diisocyanate functional groups, give polyurethanes, polyureas and polythioureas, respectively. The term “polyurethanes” as used according to the present invention encompasses these three types of polymer, namely polyurethanes themselves, polyureas and polythioureas, as well as copolymers thereof.

A first type of compound used in the preparation of the polyurethane of formula (XVIII) is a compound containing at least one unit with an amine functional group. This compound can be multifunctional, but it is preferably difunctional, i.e., in one preferred embodiment, said compound contains two labile hydrogen atoms carried e.g. by a hydroxyl, primary amine, secondary amine or thiol functional group. It is also possible to use a mixture of multifunctional and difunctional compounds in which the percentage of multifunctional compounds is low.

As indicated previously, this compound can contain more than one unit with an amine functional group, in which case it is a polymer in which the unit with an amine group is repeated.

This type of compound can be represented by one of the formulae below:

HZ-(P)ₙ-ZH

or

HZ-(P′)ₚ-ZH

in which Z, P, P′, n and p are as defined above.

Examples which may be mentioned of compounds with an functional amine group are N-methyldiethanolamine, N-tert-butyldiethanolamine and N-sulphoethyldiethanolamine.

The second compound used in the preparation of the polyurethane of formula (XVIII) is a diisocyanate of the formula:

O═C═N—R₄—N═C═O

in which R₄ is as defined above.

Examples which may be mentioned are methylenediphenyl diisocyanate, methylenecyclohexane diisocyanate, isophorone diisocyanate, toluene diisocyanate, naphthalene diisocyanate, butane diisocyanate and hexane diisocyanate.

A third compound used in the preparation of the polyurethane of formula (XVIII) is a hydrophobic compound intended for forming the terminal hydrophobic groups of the polymer of formula (XVIII).

This compound consists of a hydrophobic group and a group with a labile hydrogen, for example a hydroxyl, primary or secondary amine or thiol functional group.

By way of example, this compound can be a fatty alcohol such as, in particular, stearyl alcohol, dodecyl alcohol or decyl alcohol. If this compound contains a polymeric chain, a possible example is hydrogenated alpha-hydroxypolybutadiene.

The hydrophobic group of the polyurethane of formula (XVIII) can also result from the quaternization reaction of the tertiary amine of the compound containing at least one tertiary amine unit. The hydrophobic group is thus introduced via the quaternizing agent. This quaternizing agent is a compound of the type RQ or R′Q, in which R and R′ are as defined above and Q is a leaving group such as a halide, a sulphate, etc.

The cationic associative polyurethane can also comprise a hydrophilic sequence. This sequence is introduced via a fourth type of compound used in the preparation of the polymer. This compound can be multifunctional and is preferably difunctional. It is also possible to have a mixture in which the percentage of multifunctional compound is low.

The groups with a labile hydrogen are alcohol, primary or secondary amine or thiol groups. This compound can be a polymer terminated at the ends of the chains by one of these groups with a labile hydrogen.

Examples which may be mentioned when polymers are not involved are ethylene glycol, diethylene glycol and propylene glycol.

When a hydrophilic polymer is involved, examples which may be mentioned are polyethers, sulphonated polyesters, sulphonated polyamides or a mixture of these polymers. Preferably, the hydrophilic compound is a polyether, especially a poly(ethylene oxide) or poly(propylene oxide).

The hydrophilic group designated by Y in formula (XVIII) is optional. In fact, the units with a quaternary or protonated amine functional group may suffice to provide the necessary solubility or hydrodispersibility for this type of polymer in an aqueous solution.

Although the presence of a hydrophilic group Y is optional, it is nevertheless preferable for the cationic associative polyurethanes to contain such a group.

—(II) quaternized cellulose derivatives and polyacrylates with non-cyclic amine side groups.

The quaternized cellulose derivatives are the following in particular:

quaternized celluloses modified by groups containing at least one fatty chain, such as alkyl, arylalkyl or alkylaryl groups containing at least 8 carbon atoms, or mixtures thereof; and

quaternized hydroxyethyl celluloses modified by groups containing at least one fatty chain, such as alkyl, arylalkyl or alkylaryl groups containing at least 8 carbon atoms, or mixtures thereof.

The alkyl radicals carried by the quaternized celluloses or hydroxyethyl celluloses mentioned above preferably contain from 8 to 30 carbon atoms. The aryl radicals are preferably phenyl, benzyl, naphthyl or anthryl groups.

Examples which may be indicated of quaternized alkyl hydroxyethyl celluloses with C₈-C₃₀ fatty chains are the products QUATRISOFT LM 2000, QUATRISOFT LM-X 529-18-A®, QUATRISOFT LM-X 529-18B® (C₁₂ alkyl) and QUATRISOFT LM-X 529-8® (C₁₈ alkyl) marketed by AMERCHOL and the products CRODACEL QM®, CRODACEL QL® (C₁₂ alkyl) and CRODACEL QS® (C₁₈ alkyl) marketed by CRODA.

Amphoteric Associative Polymers

The amphoteric associative polymers are preferably selected from those containing at least one non-cyclic cationic unit. Even more particularly preferred are those prepared from or comprising 1 to 20 mol % of a monomer containing a fatty chain, preferably 1.5 to 15 mol % and more particularly 1.5 to 6 mol %, based on the total number of moles of monomers.

The preferred amphoteric associative polymers according to the invention comprise or are prepared by copolymerization of the following:

1) at least one monomer of formula (XIX) or (XX):

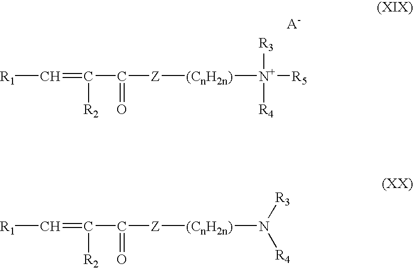

in which:

R₁ and R₂, which are identical or different, are a hydrogen atom or a methyl radical;

R₃, R₄ and R₅, which are identical or different, are a linear or branched alkyl radical having from 1 to 30 carbon atoms;

Z is an NH group or an oxygen atom;

n is an integer from 2 to 5; and

A⁻ is an anion derived from an organic or mineral acid, such as a methosulphate anion or a halide like chloride or bromide;

2) at least one monomer of formula (XXI):

R₆—CH═CR₇—COOH (XXI)

in which:

R₆ and R₇, which are identical or different, are a hydrogen atom or a methyl radical; and

3) at least one monomer of formula (XXI):

R₆—CH═CR₇—COXR₈ (XXII)

in which:

R₆ and R₇, which are identical or different, are a hydrogen atom or a methyl radical;

X is an oxygen or nitrogen atom; and

R₈ is a linear or branched alkyl radical having from 1 to 30 carbon atoms, at least one of the monomers of formula (XIX), (XX) or (XXII) containing at least one fatty chain.

The monomers of formulae (XIX) and (XX) of the present invention are preferably selected from the group comprising:

dimethylaminoethyl methacrylate, dimethylaminoethyl acrylate;

diethylaminoethyl methacrylate, diethylaminoethyl acrylate;

dimethylaminopropyl methacrylate, dimethylaminopropyl acrylate; and

dimethylaminopropylmethacrylamide, dimethylaminopropylacrylamide, these monomers optionally being quaternized, for example by a C₁-C₄ alkyl halide or a C₁-C₄ dialkyl sulphate.

More particularly, the monomer of formula (XIX) is selected from acrylamidopropyltrimethylammonium chloride and methacrylamidopropyltrimethylammonium chloride.

The monomers of formula (XXI) of the present invention are preferably selected from the group comprising acrylic acid, methacrylic acid, crotonic acid and 2-methylcrotonic acid. More particularly, the monomer of formula (XXI) is acrylic acid.

The monomers of formula (XXII) of the present invention are preferably selected from the group comprising C₁₂-C₂₂ and more particularly C₁₆-C₁₈ alkyl acrylates or methacrylates.

The monomers constituting the amphoteric polymers with a fatty chain of the invention are preferably already neutralized and/or quaternized.

The ratio of the number of cationic charges to anionic charges is preferably equal to about 1.

The amphoteric associative polymers according to the invention preferably comprise from 1 to 10 mol % of the monomer containing a fatty chain (monomer of formula (XIX), (XX) or (XXII)), preferably from 1.5 to 6 mol %.

The weight-average molecular weights of the amphoteric associative polymers according to the invention can vary from 500 to 50,000,000 and are preferably between 10,000 and 5,000,000.

The amphoteric associative polymers according to the invention can also contain other monomers such as non-ionic monomers and particularly such as C₁-C₄ alkyl acrylates or methacrylates.

Amphoteric associative polymers according to the invention are described and prepared e.g. in patent application WO 98/44012.

Acrylic acid/(meth)acrylamidopropyltrimethylammonium chloride/stearyl methacrylate terpolymers are preferred among the amphoteric associative polymers according to the invention.

The associative polymers of non-ionic type which can be used according to the invention are preferably selected from the following:

(1) celluloses modified by groups containing at least one fatty chain, of which the following may be mentioned as examples:

hydroxyethyl celluloses modified by groups containing at least one fatty chain, such as alkyl, arylalkyl or alkylaryl groups, or mixtures thereof, and in which the alkyl groups are preferably C₈-C₂₂, such as the product NATROSOL PLUS GRADE 330 CS® (C₁₆ alkyls) sold by AQUALON or the product BERMOCOLL EHM 100® sold by BEROL NOBEL; and

those modified by alkylphenol polyalkylene glycol ether groups, such as the product AMERCELL POLYMER HM-1500® (nonylphenol polyethylene glycol (15) ether) sold by AMERCHOL.

(2) hydroxypropyl guars modified by groups containing at least one fatty chain, such as the product ESAFLOR HM 22® (C₂₂ alkyl chain) sold by LAMBERTI and the products RE210-18® (C₁₄ alkyl chain) and RE205-1® (C₂₀ alkyl chain) sold by RHONE POULENC.

(3) copolymers of vinylpyrrolidone and hydrophobic monomers with a fatty chain, of which the following examples may be mentioned:

the product ANTARON V216® or GANEX V216® (vinylpyrrolidone/hexadecene copolymer) sold by ISP; and

the product ANTARON V220® or GANEX V220® (vinylpyrrolidone/eicosene copolymer) sold by ISP.

(4) copolymers of C₁-C₆ alkyl methacrylates or acrylates and amphiphilic monomers containing at least one fatty chain, for example the methyl acrylate/ethoxylated stearyl acrylate copolymer sold by GOLDSCHMIDT under the name ANTIL 208®.

(5) copolymers of hydrophilic methacrylates or acrylates and hydrophobic monomers containing at least one fatty chain, for example the polyethylene glycol methacrylate/lauryl methacrylate copolymer.

(6) polyurethanepolyethers whose chain contains hydrophilic sequences most often of a polyethoxylated nature and, at the same time, hydrophobic sequences which can be aliphatic linkages only and/or cycloaliphatic and/or aromatic linkages.

(7) polymers with an aminoplast ether skeleton possessing at least one fatty chain, such as the PURE THIX® compounds marketed by SUD-CHEMIE.

Preferably, the polyetherpolyurethanes contain at least two lipophilic hydrocarbon chains having from 6 to 30 carbon atoms and separated by a hydrophilic sequence, it being possible for the hydrocarbon chains to be pendant chains or chains at the end of the hydrophilic sequence. In particular, provision can be made for one or more pendant chains. Also, the polymer can contain a hydrocarbon chain at one or both ends of a hydrophilic sequence.

The polyetherpolyurethanes can be multisequence, particularly in the form of a tri-block. The hydrophobic sequences can be at each end of the chain (for example a tri-block copolymer with a hydrophilic central sequence) or distributed both at the ends and within the chain (for example a multisequence copolymer). These same polymers can also be in the form of grafts or star-shaped.

The non-ionic polyetherpolyurethanes with a fatty chain can be tri-block copolymers whose hydrophilic sequence is a polyethoxylated chain containing from 50 to 1000 ethylene oxide groups. The non-ionic polyetherpolyurethanes contain a urethane linkage between the hydrophilic sequences, from which the name originates.

By extension, the non-ionic polyetherpolyurethanes with a fatty chain also include those whose hydrophilic sequences are bonded to the lipophilic sequences by other chemical linkages.

As examples of non-ionic polyetherpolyurethanes with a fatty chain which can be used in the invention, it is also possible to use Rheolate 205® with a urea functional group, sold by RHEOX, or Rheolate® 208, 204 or 212, as well as Acrysol RM 184®.

Mention may also be made of the product ELFACOS T210® with a C₁₂₋₁₄ alkyl chain and the product ELFACOS T212® with a C₁₈ alkyl chain, from AKZO.

The product DW 1206B® with a C₂₀ alkyl chain and a urethane linkage, marketed by ROHM & HAAS at a solids concentration of 20% in water, can also be used.

It is further possible to use solutions or dispersions of these polymers, especially in water or an aqueous-alcoholic medium. Examples of such polymers which may be mentioned are Rheolates 255, Rheolate® 278 and Rheolate® 244, sold by RHEOX. The products DW 1206F and DW 1206J, marketed by ROHM & HAAS, can also be used.

The polyetherpolyurethanes which can be used according to the invention are particularly those described in the article by G. Formum, J. Bakke and Fk. Hansen—Colloid Polym. Sci. 271, 380-389 (1993).

It is even more particularly preferable to use a polyetherpolyurethane that can be obtained by the polycondensation of at least three compounds comprising (i) at least one polyethylene glycol containing from 150 to 180 mol of ethylene oxide, (ii) stearyl alcohol or decyl alcohol, and (iii) at least one diisocyanate.

Such polyetherpolyurethanes are sold especially by ROHM & HAAS under the names Aculyn 46® and Aculyn 44® [ACULYN 46® is a polycondensation product of polyethylene glycol containing 150 or 180 mol of ethylene oxide, stearyl alcohol and methylene-bis(4-cyclohexyl isocyanate) (SMD) at a concentration of 15% by weight in a matrix of maltodextrin (4%) and water (81%); ACULYN 44® is a polycondensation product of polyethylene glycol containing 150 or 180 mol of ethylene oxide, decyl alcohol and methylene-bis(4-cyclohexyl isocyanate) (SMDI) at a concentration of 35% by weight in a mixture of propylene glycol (39%) and water (26%)].

In a third preferred embodiment, the composition according to the present invention also contains at least one surfactant.

The surfactants suitable for carrying out the present invention are the following in particular:

(i) Anionic Surfactant(s):

Examples which may be mentioned in particular of anionic surfactants which can be used, by themselves or in mixtures, within the framework of the present invention (without implying a limitation) are salts (particularly alkali metal salts, especially sodium salts, ammonium salts, amine salts, amino alcohol salts or magnesium salts) of the following compounds: alkylsulphates, alkyl-ether-sulphates, alkylamido-ether-sulphates, alkylaryl-polyether-sulphates and monoglyceride-sulphates; alkylsulphonates, alkylphosphates, alkylamidesulphonates, alkylaryl-sulphonates, α-olefinsulphonates and paraffinsulphonates; alkyl(C₆-C₂₄)sulphosuccinates, alkyl(C₆-C₂₄)-ether-sulphosuccinates and alkyl(C₆-C₂₄)amidesulphosuccinates; alkyl(C₆-C₂₄)sulphoacetates; and acyl(C₆-C₂₄)sarcosinates and acyl(C₆-C₂₄)glutamates. It is also possible to use carboxylic acid esters of alkyl(C₆-C₂₄)polyglycosides, such as alkylglucoside citrates, alkylpolyglycoside tartrates and alkylpolyglycoside sulphosuccinates, alkylsulphosuccinamates, acylisethionates and N-acyltaurates, the alkyl or acyl radical in all these various compounds preferably containing from 12 to 20 carbon atoms and the aryl radical preferably being a phenyl or benzyl group. The following may also be mentioned among other anionic surfactants which can be used: fatty acid salts such as the salts of oleic, ricinoleic, palmitic and stearic acids and the acids of copra oil or hydrogenated copra oil; and acyllactylates in which the acyl radical contains 8 to 20 carbon atoms. It is also possible to use alkyl-D-galactosideuronic acids and their salts, polyalkoxylated alkyl(C₆-C₂₄)-ether-carboxylic acids, polyalkoxylated alkyl(C₆-C₂₄)aryl-ether-carboxylic acids, polyalkoxylated alkyl(C₆-C₂₄)amido-ether-carboxylic acids and their salts, especially those containing from 2 to 50 alkylene oxide groups, particularly ethylene oxide groups, and mixtures thereof.

(ii) Non-Ionic Surfactant(s):

The non-ionic surfactants are again compounds that are well known per se (cf. especially “Handbook of Surfactants” by M. R. PORTER, published by Blackie & Son (Glasgow and London), 1991, pp 116-178) and their nature is not critical within the framework of the present invention. Thus they can be selected especially (without implying a limitation) from alcohols, alpha-diols and polyethoxylated or polypropoxylated alkylphenols having a fatty chain containing e.g. 8 to 18 carbon atoms, it being possible for the number of ethylene oxide or propylene oxide groups to range from 2 to 50 in particular. Other compounds which may be mentioned are ethylene oxide/propylene oxide copolymers and condensation products of ethylene oxide and propylene oxide with fatty alcohols; polyethoxylated fatty amides preferably containing from 2 to 30 mol of ethylene oxide, and polyglycerolated fatty amides containing an average of 1 to 5 glycerol groups and particularly 1.5 to 4 glycerol groups; ethoxylated sorbitan fatty acid esters containing from 2 to 30 mol of ethylene oxide; and sucrose fatty acid esters, polyethylene glycol fatty acid esters, alkylpolyglycosides, N-alkylglucamine derivatives, and amine oxides such as alkyl(C₁₀-C₁₄)amine oxides or N-acylaminopropylmorpholine oxides.

(iii) Amphoteric or Zwitterionic Surfactant(s):

The amphoteric or zwitterionic surfactants, which are not of a critical nature within the framework of the present invention, can be especially (without implying a limitation) aliphatic secondary or tertiary amine derivatives in which the aliphatic radical is a linear or branched chain containing 8 to 18 carbon atoms and at least one water-solubilizing anionic group (e.g. carboxylate, sulphonate, sulphate, phosphate or phosphonate); other compounds which may be mentioned are alkyl(C₈-C₂₀)betaines, sulphobetaines, alkyl(C₈-C₂₀)amidoalkyl(C₁-C₆)betaines or alkyl(C₈-C₂₀)amidoalkyl(C₁-C₆)sulphobetaines.

Products which may be mentioned among the amine derivatives are those sold under the name MIRANOL, such as the products described in patents U.S. Pat. No. 2,528,378 and U.S. Pat. No. 2,781,354 and classified in the CTFA dictionary, 3rd edition, 1982, under the names amphocarboxyglycinates and amphocarboxypropionates, which have the following respective structures:

R₂—CONHCH₂CH₂—N(R₃)(R₄)(CH₂COO—)

in which:

R₂ is an alkyl radical of an acid R₂—COOH present in hydrolysed copra oil, or a heptyl, nonyl or undecyl radical;

R₃ is a beta-hydroxyethyl group; and

R₄ is a carboxymethyl group; and

R₂′—CONHCH₂CH₂—N(B)(C)

in which:

B is —CH₂CH₂OX′;

C is —(CH₂), —Y′, where z=1 or 2;

X′ is the group —CH₂CH₂—COOH or a hydrogen atom;

Y′ is —COOH or the radical —CH₂—CHOH—SO₃H; and

R₂′ is an alkyl radical of an acid R₉13 COOH present in copra oil or in hydrolysed linseed oil, or an alkyl radical, especially a C₇, C₉, C₁₁ or C₁₃ alkyl radical, a C₁₇ alkyl radical or its iso form, or an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

An example which may be mentioned is the cocoamphodiacetate marketed under the trade name MIRANOL® C2M concentre by RHODIA CHIMIE.

(iv) Cationic Surfactants:

The following may be mentioned in particular (without implying a limitation) among the cationic surfactants: salts of optionally polyalkoxylated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic character.

The amounts of surfactants present in the composition according to the invention can vary from 0.01 to 40% and preferably from 0.5 to 30% of the total weight of the composition.

The composition of the present invention can also comprise one or more additional oxidation bases conventionally used in oxidation dyeing, other than the paraphenylenediamines of formula I. By way of example, these additional oxidation bases are selected from phenylenediamines, bis-phenylalkylenediamines, paraminophenols, orthoaminophenols, heterocyclic bases other than heterocyclic paraphenylenediamines, and their addition salts.

Examples of paraphenylenediamines which may be mentioned are paraphenylenediamine, paratoluylenediamine, 2-chloroparaphenylenediamine, 2,3-dimethylparaphenylenediamine, 2,6-dimethylparaphenylenediamine, 2,6-diethylparaphenylenediamine, 2,5-dimethylparaphenylenediamine, N,N-dimethylparaphenylenediamine, N,N-diethylparaphenylenediamine, N,N-dipropylparaphenylenediamine, 4-amino-N,N-diethyl-3-methylaniline, N,N-bis(β-hydroxyethyl)-paraphenylenediamine, 4-N,N-bis(P-hydroxyethyl)amino-2-methylaniline, 4-N,N-bis(β-hydroxyethyl)amino-2-chloroaniline, 2-1-hydroxyethylparaphenylenediaamine, 2-fluoroparaphenylenediamine, 2-isopropylparaphenylenediamine, N-(β-hydroxypropyl)paraphenylenediamine, 2-hydroxymethylparaphenylenediamine, N,N-dimethyl-3-methylparaphenylenediamine, N,N-(ethyl,β-hydroxyethyl)paraphenylenediamine, N-(β,γ-dihydroxypropyl)paraphenylenediamine, N-(4′-aminophenyl)paraphenylenediamine, N-phenylparaphenylenediamine, 2-β-hydroxyethoxyparaphenylenediamine, 2-O-acetylaminoethoxyparaphenylenediamine, N-(β-methoxyethyl)paraphenylenediamine, 4-aminophenylpyrrolidine, 2-thienylparaphenylenediamine, 2-β-hydroxyethylamino-5-aminotoluene, 3-hydroxy-1-(4′-aminophenyl)pyrrolidine and their addition salts with an acid.

The following are particularly preferred among the paraphenylenediamines mentioned above: paraphenylenediamine, paratoluylenediamine, 2-isopropylparaphenylenediamine, 2-β-hydroxyethylparaphenylenediamine, 2-β-hydroxyethoxyparaphenylenediamine, 2,6-dimethylparaphenylenediamine, 2,6-diethylparaphenylenediamine, 2,3-dimethylparaphenylenediamine, N,N-bis(β-hydroxyethyl)paraphenylenediamine, 2-chloroparaphenylenediamine, 2-β-acetylaminoethoxyparaphenylenediamine and their addition salts with an acid.

Examples of bis-phenylalkylenediamines which may be mentioned are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis-(ethyl)-N,N′-bis(4′-amino,3′-methylphenyl)ethylenediamine, 1,8-bis(2,5-diaminophenoxy)-3,6-dioxaoctane and their addition salts.

Examples of paraminophenols which may be mentioned are paraminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol and their addition salts with an acid.

Examples of orthoaminophenols which may be mentioned are 2-aminophenol, 2-amino-5-methylphenol, 2-amino-6-methylphenol, 5-acetamido-2-aminophenol and their addition salts.

Examples of heterocyclic bases which may be mentioned are pyridine derivatives, pyrimidine derivatives and pyrazole derivatives.

Compounds which may be mentioned among the pyridine derivatives are those described e.g. in patents GB 1 026 978 and GB 1 153 196, such as 2,5-diaminopyridine, 2-(4-methoxyphenyl)amino-3-aminopyridine, 3,4-diaminopyridine and their addition salts.

Other pyridine oxidation bases which are useful in the present invention are the 3-aminopyrazolo[1,5-a]pyridine oxidation bases or their addition salts described e.g. in patent application FR 2 801 308. Examples which may be mentioned are pyrazolo[1,5-a]pyridin-3-ylamine, 2-acetylaminopyrazolo[1,5-a]pyridin-3-ylamine, 2-morpholin-4-ylpyrazolo[1,5-a]pyridin-3-ylamine, 3-aminopyrazolo[1,5-a]pyridine-2-carboxylic acid, 2-methoxypyrazolo[1,5-a]pyridin-3-ylanine, (3-aminopyrazolo[1,5-a]pyridin-7-yl)methanol, 2-(3-aminopyrazolo[1,5-a]pyridin-5-yl)ethanol, 2-(3-aminopyrazolo[1,5-a]pyridin-7-yl)ethanol, (3-aminopyrazolo[1,5-a]pyridin-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyridin-3-ylamine, pyrazolo[1,5-a]pyridine-3,5-diaamine, 5-morpholin-4-ylpyrazolo[1,5-a]pyridin-3-ylamine, 2-[(3-aminopyrazolo[1,5-a]pyridin-5-yl)(2-hydroxyethyl)amino]ethanol, 2-[(3-aminopyrazolo[1,5-a]pyridin-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol and their addition salts.

Compounds which may be mentioned among the pyrimidine derivatives are those described e.g. in patents DE 2 359 399, JP 88-169571, JP 05-63124 and EP 0 770 375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and, if a tautomeric equilibrium exists, their tautomeric forms.

Compounds which may be mentioned among the pyrazole derivatives are those described in patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)-pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and their addition salts.

The additional oxidation base(s) in the composition of the invention are generally present in an amount ranging from about 0.001 to 20% by weight, based on the total weight of the dyeing composition, and preferably ranging from 0.005 to 6%.

The composition according to the invention preferably contains one or more additional couplers conventionally used for the dyeing of keratin fibres. The following may be mentioned in particular among these couplers: metaphenylenediamines, metadiphenols, naphthalene couplers, heterocyclic couplers and their addition salts.

Examples which may be mentioned are 1,3-dihydroxybenzene, 1,3-dihydroxy-2-methylbenzene, 4-chloro-1,3-dihydroxybenzene, 2,4-diamino-1-(β-hydroxyethoxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene and their addition salts.

The coupler(s) are generally present in the composition of the present invention in an amount ranging from about 0.001 to 20% by weight, based on the total weight of the dyeing composition, and preferably ranging from 0.005 to 6%.

In general terms, the addition salts of the oxidation bases and couplers which can be used within the framework of the invention are selected especially from addition salts with an acid, such as hydrochlorides, hydrobromides, sulphates, citrates, succinate, tartrates, lactates, tosylates, benzenesulphonates, phosphates and acetates, and addition salts with a base such as sodium hydroxide, potassium hydroxide, aqueous ammonia, an amine or an alkanolamine.

The dyeing composition according to the invention can also contain one or more direct dyestuffs which can be selected in particular from neutral, acidic or cationic nitro dyestuffs of the benzene series, neutral, acidic or cationic azo direct dyestuffs, neutral, acidic or cationic quinone direct dyestuffs, particularly anthraquinone direct dyestuffs, azine direct dyestuffs, triarylmethane direct dyestuffs, indoamine direct dyestuffs and natural direct dyestuffs.

The following compounds may be mentioned, without implying a limitation, among the benzene direct dyestuffs which can be used according to the invention:

1,4-diamino-2-nitrobenzene;

1-amino-2-nitro-4-β-hydroxyethylaminobenzene;

1-amino-2-nitro-4-bis(β-hydroxyethyl)aminobenzene;

1,4-bis(β-hydroxyethylamino)-2-nitrobenzene;

1-β-hydroxyethylamino-2-nitro-4-bis(β-hydroxyethylamino)benzene;

1-β-hydroxyethylamino-2-nitro-4-aminobenzene;

1-β-hydroxyethylamino-2-nitro-4-(ethyl)(β-hydroxyethyl)aminobenzene;

1-amino-3-methyl-4-β-hydroxyethylamino-6-nitrobenzene;

1-amino-2-nitro-4-p-hydroxyethylamino-5-chlorobenzene;

1,2-diamino-4-nitrobenzene;

1-amino-2-β-hydroxyethylamino-5-nitrobenzene;

1,2-bis(β-hydroxyethylamino)-4-nitrobenzene;

1-amino-2-tris(hydroxymethyl)methylamino-5-nitrobenzene;

1-hydroxy-2-amino-5-nitrobenzene;

1-hydroxy-2-amino-4-nitrobenzene;

1-hydroxy-3-nitro-4-aminobenzene;

1-hydroxy-2-amino-4,6-dinitrobenzene;

1-β-hydroxyethoxy-2-O-hydroxyethylamino-5-nitrobenzene;

1-methoxy-2-β-hydroxyethylamino-5-nitrobenzene;

1-β-hydroxyethoxy-3-methylamino-4-nitrobenzene;

1-β,γ-dihydroxypropoxy-3-methylamino-4-nitrobenzene;

1-β-hydroxyethylamino-4-β,γ-dihydroxypropoxy-2-nitrobenzene;

1-β,γ-dihydroxypropylamino-4-trifluoromethyl-2-nitrobenzene;

1-β-hydroxyethylamino-4-trifluoromethyl-2-nitrobenzene;

1-β-hydroxyethylamino-3-methyl-2-nitrobenzene;

1-β-aminoethylamino-5-methoxy-2-nitrobenzene;

1-hydroxy-2-chloro-6-ethylamino-4-nitrobenzene;

1-hydroxy-2-chloro-6-amino-4-nitrobenzene;

1-hydroxy-6-bis(β-hydroxyethyl)amino-3-nitrobenzene;

1-β-hydroxyethylamino-2-nitrobenzene; and

1-hydroxy-4-β-hydroxyethylamino-3-nitrobenzene.

The cationic azo direct dyestuffs described in patent applications WO 95/15144, WO 95/01772 and EP-714 954, the content of which forms an integral part of the invention, may be mentioned among the azo direct dyestuffs which can be used according to the invention.

The following dyestuffs may be mentioned very particularly among these compounds:

1,3-dimethyl-2-[[4-(dimethylamino)phenyl]azo]-1H-imidazolium chloride;

1,3-dimethyl-2-[(4-aminophenyl)azo]-1H-imidazolium chloride; and

1-methyl-4-[(methylphenylhydrazono)methyl]pyridinium methylsulphate.

The following dyestuffs described in COLOUR INDEX INTERNATIONAL, 3rd edition, may also be mentioned among the azo direct dyestuffs:

Disperse Red 17;

Acid Yellow 9;

Acid Black 1;

Basic Red 22;

Basic Red 76;

Basic Yellow 57;

Basic Brown 16;

Acid Yellow 36;

Acid Orange 7;

Acid Red 33;

Acid Red 35;

Basic Brown 17;

Acid Yellow 23;

Acid Orange 24; and

Disperse Black 9.

1-(4′-Aminodiphenylazo)-2-methyl-4-bis(β-hydroxyethyl)aminobenzene and 4-hydroxy-3-(2-methoxyphenylazo)-1-naphthalenesulphonic acid may also be mentioned.

The following dyestuffs:

Disperse Red 15;

Solvent Violet 13;

Acid Violet 43;

Disperse Violet 1;

Disperse Violet 4;

Disperse Blue 1;

Disperse Violet 8;

Disperse Blue 3;

Disperse Red 11;

Acid Blue 62;

Disperse Blue 7;

Basic Blue 22;

Disperse Violet 15; and

Basic Blue 99

and the following compounds:

1-N-methylmorpholiniumpropylamino-4-hydroxyanthraquinone;

1-aminopropylamino-4-methylaminoanthraquinone;

1-aminopropylaminoanthraquinone;

-5-β-hydroxyethyl-1,4-diaminoanthraquinone;

-2-aminoethylaminoanthraquinone; and

1,4-bis(β,γ-dihydroxypropylamino)anthraquinone

may be mentioned among the quinone direct dyestuffs.

The following compounds may be mentioned among the azine dyestuffs:

Basic Blue 17; and

Basic Red 2.

The following compounds may be mentioned among the triarylmethane dyestuffs which can be used according to the invention:

Basic Green 1;

Acid Blue 9;

Basic Violet 3;

Basic Violet 14;

Basic Blue 7;

Acid Violet 49;

Basic Blue 26; and

Acid Blue 7.

The following compounds may be mentioned among the indoamine dyestuffs which can be used according to the invention:

2-β-hydroxyethylamino-5-[bis(P-4′-hydroxyethyl)amino]anilino-1,4-benzoquinone;

2-β-hydroxyethylamino-5-(2′-methoxy-4′-amino)anilino-1,4-benzoquinone;

-3-N-(2′-chloro-4′-hydroxy)phenylacetylamino-6-methoxy-1,4-benzoquinone imine;

3-N-(3′-chloro-4′-methylamino)phenylureido-6-methyl-1,4-benzoquinone imine; and

3-[4′-N-(ethyl,carbamylmethyl)amino]phenylureido-6-methyl-1,4-benzoquinone imine.

The following may be mentioned among the natural direct dyestuffs which can be used according to the invention: lawsone, juglone, alizarin, purpurin, carminic acid, kermesic acid, purpurogallin, protocatechaldehyde, indigo, isatin, curcumin, spinulosin and apigenidine. It is also possible to use extracts or decoctions containing these natural dyestuffs, and especially poultices or extracts based on henna.

The direct dyestuff(s) preferably represent from about 0.001 to 20% by weight, based on the total weight of the ready-to-use composition, and even more preferably from about 0.005 to 10% by weight.

The composition according to the invention can also contain at least one hydroxylated solvent such as, in particular, ethanol, propylene glycol, glycerol, a polyol monoether or benzyl alcohol.

It can also contain a non-hydroxylated solvent.

Preferably, the hydroxylated solvents and non-hydroxylated solvents are present in proportions preferably of between about 1 and 40% by weight, based on the total weight of the dyeing composition, and even more preferably of between about 5 and 30% by weight.

The dyeing composition according to the invention can also contain various adjuvants conventionally used in hair dyeing compositions, such as antioxidants, penetrating agents, sequestering agents, perfumes, buffers, dispersants, conditioners, for example modified or unmodified, volatile or non-volatile silicones, film-forming agents, ceramides, preservatives and opacifying agents.

The above adjuvants are each generally present in an amount of between 0.01 and 20% by weight, based on the weight of the composition.

Of course, those skilled in the art will take care to choose this or these possible complementary compounds in such a way that the advantageous properties intrinsically attached to the oxidation dyeing composition according to the invention are unaffected or substantially unaffected by the addition(s) envisaged.

The pH of the dyeing composition according to the invention is generally between about 3 and 12 and preferably between about 5 and 11. It can be adjusted to the desired value with acidifying or alkalizing agents normally used in the dyeing of keratin fibres, or with conventional buffer systems.

Examples of acidifying agents which may be mentioned are mineral or organic acids such as hydrochloric acid, orthophosphoric acid, sulphuric acid, carboxylic acids like acetic acid, tartaric acid, citric acid and lactic acid, and sulphonic acids.

Examples of alkalizing agents which may be mentioned are aqueous ammonia, alkali metal carbonates, alkanolamines such as mono-, di- and triethanolamines and their derivatives, sodium and potassium hydroxides and the compounds of formula (XXII) below:

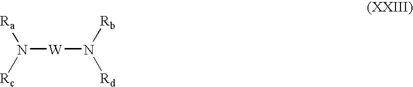

in which W is a propylene radical optionally substituted by a hydroxyl group or a C₁-C₄ alkyl radical, and Rₐ, R_{b}, R_{c} and R_{d}, which are identical or different, are a hydrogen atom or a C₁-C₄ alkyl or C₁-C₄-hydroxyalkyl radical.

The dyeing composition according to the invention can take a variety of forms such as liquids, creams, gels or any other form appropriate for the dyeing of keratin fibres and especially human hair.

The process of the present invention is a process in which the composition according to the present invention, as defined above, is applied to the fibres and the colour is developed with an oxidizing agent. The colour can be developed at acidic, neutral or alkaline pH and the oxidizing agent can be added to the composition of the invention at the time of use; alternatively, it can be introduced from an oxidizing composition containing it, applied simultaneously with the composition of the invention or afterwards.

In one particular embodiment, the composition according to the present invention is mixed, preferably at the time of use, with a composition containing at least one oxidizing agent in a medium appropriate for dyeing, said oxidizing agent being present in a sufficient amount to develop a colouration. The mixture obtained is then applied to the keratin fibres. After a waiting time of about 3 to 50 minutes and preferably of about 5 to 30 minutes, the keratin fibres are rinsed, shampooed, rinsed again and then dried.

The oxidizing agents conventionally used for the oxidation dyeing of keratin fibres are e.g. hydrogen peroxide, urea peroxide, alkali metal bromates, per-salts such as perborates and persulphates, per-acids and oxidase enzymes, among which there may be mentioned peroxidases, oxido-reductases containing 2 electrons, such as uricases, and oxygenases containing 4 electrons, such as laccases. Hydrogen peroxide is particularly preferred.

The oxidizing composition can also contain various adjuvants conventionally used in hair dyeing compositions, such as those defined above.

The pH of the oxidizing composition containing the oxidizing agent is such that, after mixing with the dyeing composition, the pH of the resulting composition applied to the keratin fibres preferably varies between about 3 and 12 and even more preferably between 5 and 11. It can be adjusted to the desired value with acidifying or alkalizing agents normally used in the dyeing of keratin fibres, such as those defined above.

The ready-to-use composition which is finally applied to the keratin fibres can take a variety of forms such as liquids, creams, gels or any other form appropriate for the dyeing of keratin fibres and especially human hair.

The invention further relates to a multicompartment dyeing device, or dyeing kit, in which a first compartment contains the dyeing composition defined above and a second compartment contains an oxidizing composition. This device can be equipped with a means of dispensing the desired mixture onto the hair, such as the devices described in patent FR-2 586 913 in the name of the Applicant.

Using this device, it is possible to dye the keratin fibres by means of a process which comprises mixing a dyeing composition according to the invention with an oxidizing agent as defined above, and applying the resulting mixture to the keratin fibres for a sufficient time to develop the desired colouration.

### DETAILED DESCRIPTION

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Dyeing Composition (Expressed in Grams):

| | |
|---|---|
| Oleyl alcohol | 6 |
| Oleic acid | 3 |
| Oleyl alcohol polyglycerolated with 2 mol of glycerol | 6 |
| Oleyl alcohol polyglycerolated with 6 mol of glycerol | 6 |
| Diethylaminopropyl laurylaminosuccinamate, sodium salt | 3 |
| Oleylamine ethoxylated with 2 mol of ethylene oxide | 7 |
| Alkyl-ether-carboxylic acid monoethanolamide containing 2 mol | 10 |
| of ethylene oxide | |
| Ammonium acetate | 20 |
| Hexylene glycol | 20 |
| Sucrose | 1 |
| Reducing agents, antioxidants | 0.915 |
| Sequestering agents | 1 |
| 1,3-Dihydroxybenzene (resorcinol) | 0.085 |
| [1-(4-Aminophenyl)pyrrolidin-3-yl]trimethylammonium, | 1.0 |
| chloride | |
| 2-Methyl-5-aminophenol | 0.5 |
| Perfume | qs |
| Aqueous ammonia (containing 20.5% of ammonia) | 10.2 |
| Demineralized water qsp | 100 |

At the time of use, this composition is mixed with an equal weight of an oxidizing milk containing 6% of hydrogen peroxide. The resulting mixture is applied for 30 minutes to grey hair containing 90% of white hair. A brownish-violet colouration is obtained on this hair after rinsing, shampooing and drying.

### Example 2

Dyeing Composition (Expressed in Grams):

| | |
|---|---|
| Oleyl alcohol | 4 |
| Oleic acid | 5 |
| Oleyl alcohol polyglycerolated with 2 mol of glycerol | 4 |
| Lauryl alcohol polyglycerolated with 4 mol of glycerol | 3.6 |
| Amide of colza acids, ethoxylated with 4 mol of ethylene oxide | 8 |
| Oleylamine ethoxylated with 2 mol of ethylene oxide | 4 |
| Decyl alcohol ethoxylated with 3 mol of ethylene oxide | 2.7 |
| Ethyl alcohol | 7.45 |
| Propylene glycol | 15 |
| Glucose | 1 |
| Reducing agents, antioxidants | 0.63 |
| Sequestering agent | 1 |
| 3-[1-(4-Aminophenyl)pyrrolidin-3-yl]-1-methyl-3H-imidazol-1- | 1.0 |
| ium, chloride | |
| 5-N-(β-hydroxyethyl)amino-2-methylphenol | 0.5 |
| Pure monoethanolamine | 2 |
| Perfume | qs |
| Aqueous ammonia (containing 20.5% of ammonia) | 10 |
| Demineralized water qsp | 100 |

At the time of use, this composition is mixed with an equal weight of an oxidizing milk containing 6% of hydrogen peroxide. The resulting mixture is applied for 30 minutes to grey hair containing 90% of white hair. A deep purple colouration is obtained on this hair after rinsing, shampooing and drying.

What is claimed is:

1. A composition for dyeing keratin fibres, comprising, in an appropriate dyeing medium, at least one cationic tertiary paraphenylenediamine containing a pyrrolidine ring, and at least one carbohydrate selected from monosaccharides, disaccharides and mixtures thereof.

2. The composition of claim 1, wherein the cationic tertiary para-phenylenediamine comprises formula I: 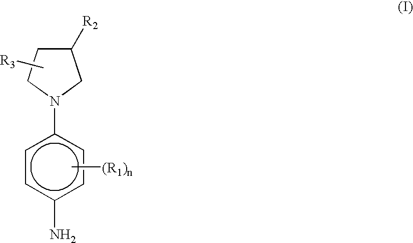
in which:
n varies from 0 to 4, it being understood that if n is greater than or equal to 2, the radicals R₁ can be identical or different;
R₁ is a halogen atom; a saturated or unsaturated, aliphatic or alicyclic C₁-C₆ hydrocarbon chain, it being possible for the chain to contain one or more oxygen, nitrogen, silicon or sulphur atoms or an SO₂ group and to be substituted by one or more hydroxyl or amino radicals; or an onium radical Z, the radical R₁ containing neither a peroxide linkage nor diazo, nitro or nitroso radicals;
R₂ is an onium radical Z or a radical —X—C═NR₈—NR₉R₁₀, in which X is an oxygen atom or a radical —NR₁₁, and R₈, R₉, R₁₀ and R₁₁ are a hydrogen atom, a C₁-C₄ alkyl radical or a C₁-C₄ hydroxyalkyl radical; and
R₃ is a hydrogen atom or a hydroxyl radical.

3. The composition of claim 2, wherein the cationic tertiary para-phenylenediamine is such that n is equal to 0.

4. The composition of claim 2, wherein the cationic tertiary paraphenylenediamine is such that n is equal to 1 and R₁ is selected from the group comprising a halogen atom and a saturated or unsaturated, aliphatic or alicyclic C₁-C₆ hydrocarbon chain, it being possible for one or more carbon atoms to be replaced by an oxygen, nitrogen, silicon or sulphur atom or by an SO₂ group, the radical R₁ containing neither a peroxide linkage nor diazo, nitro or nitroso radicals.

5. The composition of claim 2, wherein the cationic tertiary paraphenylenediamine is such that R₁ is selected from chlorine, bromine and C₁-C₄ alkyl, C₁-C₄ hydroxyalkyl, C₁-C₄ aminoalkyl, C₁-C₄ alkoxy and C₁-C₄ hydroxyalkoxy radicals.

6. The composition of claim 5, wherein the cationic tertiary paraphenylenediamine is such that R₁ is selected from methyl, hydroxymethyl, 2-hydroxyethyl, 1,2-dihydroxyethyl, methoxy, isopropoxy and 2-hydroxyethoxy radicals.

7. The composition of claim 2, wherein the cationic tertiary paraphenylenediamine is such that R₂ is the onium radical Z of formula (II): 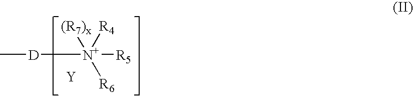
in which:
D is a single bond or a linear or branched C₁-C₁₄ alkylene chain capable of containing one or more heteroatoms selected from oxygen, sulphur and nitrogen, capable of being substituted by one or more hydroxyl, C₁-C₆ alkoxy or amino radicals and capable of carrying one or more ketone groups;
R₄, R₅ and R₆, taken separately, are a C₁-C₁₅ alkyl radical; a C₁-C₆ monohydroxyalkyl radical; a C₂-C₆ polyhydroxyalkyl radical; a C₁-C₆ alkoxy(C₁-C₆)alkyl radical; an aryl radical; a benzyl radical; a C₁-C₆ amidoalkyl radical; a C₁-C₆ trialkyl(C₁-C₆)silanalkyl radical; a C₁-C₆ aminoalkyl radical; or a C₁-C₆ aminoalkyl radical in which the amine is monosubstituted or disubstituted by a C₁-C₄ alkyl, alkyl(C₁-C₆)carbonyl, amido or alkyl(C₁-C₆)sulphonyl radical; or
R₄, R₅ and R₆, taken together in pairs, form, with the nitrogen atom to which they are attached, a 4-, 5-, 6- or 7-membered saturated carbon-containing ring capable of containing one or more heteroatoms, it being possible for the cationic ring to be substituted by a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a C₁-C₆ trialkyl(C₁-C₆)silanalkyl radical, an amido radical, a carboxyl radical, an alkyl(C₁-C₆)carbonyl radical, a thio radical (—SH), a C₁-C₆ thioalkyl radical (—R—SH), an alkyl(C₁-C₆)thio radical, an amino radical or an amino radical monosubstituted or disubstituted by an alkyl(C₁-C₆), alkyl(C₁-C₆)carbonyl, amido or alkyl(C₁-C₆)sulphonyl radical;
R₇ is a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carboxyalkyl radical; a C₁-C₆ carbamylalkyl radical; a C₁-C₆ trifluoroalkyl radical; a C₁-C₆ trialkyl(C₁-C₆)silanalkyl radical; a C₁-C₆ sulphonamidoalkyl radical; a C₁-C₆ alkyl(C₁-C₆)carboxyalkyl radical; a C₁-C₆ alkyl(C₁-C₆)sulphinylalkyl radical; a C₁-C₆ alkyl(C₁-C₆)sulphonylalkyl radical; a C₁-C₆ alkyl(C₁-C₆)carbonylalkyl radical; a C₁-C₆ N-alkyl(C₁-C₆)carbamylalkyl radical; or a C₁-C₆ N-alkyl(C₁-C₆)sulphonamidoalkyl radical;
x is 0 or 1:
if x=0, the linking arm is attached to the nitrogen atom carrying the radicals R₄ to R₆;
if x=1, two of the radicals R₄ to R₆ form a 4-, 5-, 6- or 7-membered saturated ring together with the nitrogen atom to which they are attached, and D is bonded to a carbon atom of the saturated ring; and
Y is a counterion.

8. The composition of claim 7, wherein the cationic tertiary paraphenylenediamine is such that R₂ has formula II in which x is equal to 0 and R₄, R₅ and R₆, taken separately, are preferably selected from a C₁-C₆ alkyl radical; a C₁-C₄ monohydroxyalkyl radical; a C₂-C₄ polyhydroxyalkyl radical; a C₁-C₄ alkoxy-(C₁-C₆)alkyl radical; a C₁-C₆ amidoalkyl radical; and a C₁-C₆ trialkyl(C₁-C₆)silanalkyl radical, or R₄ and R₅ together form an azetidine, pyrrolidine, piperidine, piperazine or morpholine ring, R₆ being selected in this case from a C₁-C₆ alkyl radical; a C₁-C₆ monohydroxyalkyl radical; a C₂-C₆ polyhydroxyalkyl radical; a C₁-C₆ aminoalkyl radical; an aminoalkyl radical monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carboxyalkyl radical; a C₁-C₆ alkyl(C₁-C₆)carbonylalkyl radical; and a C₁-C₆ N-alkyl(C₁-C₆)carbamylalkyl radical.

9. The composition of claim 7, wherein the cationic tertiary paraphenylenediamine is such that R₂ has formula II in which x is equal to 1 and R₇ is selected from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carboxyalkyl radical; a C₁-C₆ alkyl(C₁-C₆)carbonylalkyl radical; and a C₁-C₆ N-alkyl(C₁-C₆)carbamylalkyl radical; and R₄ and R₅ together form an azetidine, pyrrolidine, piperidine, piperazine or morpholine ring, R₆ being selected in this case from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carboxyalkyl radical; a C₁-C₆ alkyl(C₁-C₆)carbonylalkyl radical; and a C₁-C₆ N-alkyl(C₁-C₆)carbamylalkyl radical.

10. The composition of claim 2, wherein the cationic tertiary paraphenylenediamine is such that D is a single bond or an alkylene chain capable of being substituted.

11. The composition of claim 7, wherein the cationic tertiary paraphenylenediamine is such that R₂ is a trialkylammonium radical.

12. The composition of claims 2, wherein the cationic tertiary paraphenylenediamine is such that R₂ is the onium radical Z of formula III: 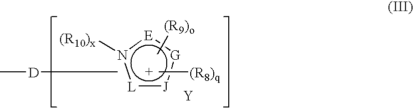
in which:
D is a single bond or a linear or branched C₁-C₁₄ alkylene chain capable of containing one or more heteroatoms selected from oxygen, sulphur and nitrogen, capable of being substituted by one or more hydroxyl, C₁-C₆ alkoxy or amino radicals and capable of carrying one or more ketone functional groups;
the vertices E, G, J and L, which are identical or different, are a carbon, oxygen, sulphur or nitrogen atom so as to form a pyrrole, pyrazole, imidazole, triazole, oxazole, isooxazole, thiazole or isothiazole ring;
q is an integer between 0 and 4 inclusive;
o is an integer between 0 and 3 inclusive;
q+o is an integer between 0 and 4;
the radicals R₈, which are identical or different, are a halogen atom; a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a carboxyl radical; a C₁-C₆ alkylcarbonyl radical; a thio radical; a C₁-C₆ thioalkyl radical; an alkyl(C₁-C₆)thio radical; an amino radical; an amino radical monosubstituted or disubstituted by an alkyl(C₁-C₆), alkyl(C₁-C₆)carbonyl, amido or alkyl(C₁-C₆)sulphonyl radical; a C₁-C₆ monohydroxyalkyl radical; or a C₂-C₆ polyhydroxyalkyl radical, it being understood that the radicals R₈ are carried by a carbon atom;
the radicals R₉, which are identical or different, are a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; a C₁-C₆ carbamylalkyl radical; a C₁-C₆ alkyl(C₁-C₆)carboxyalkyl radical; or a benzyl radical, it being understood that the radicals R₉ are carried by a nitrogen;
R₁₀ is a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is substituted by an alkyl(C₁-C₆), alkyl(C₁-C₆)carbonyl, amido or alkyl(C₁-C₆)sulphonyl radical; a C₁-C₆ carboxyalkyl radical; a C₁-C₆ carbamylalkyl radical; a C₁-C₆ trifluoroalkyl radical; a C₁-C₆ trialkyl(C₁-C₆)silanalkyl radical; a C₁-C₆ sulphonamidoalkyl radical; a C₁-C₆ alkyl(C₁-C₆)carboxyalkyl radical; a C₁-C₆ alkyl(C₁-C₆)sulphinylalkyl radical; a C₁-C₆ alkyl(C₁-C₆)sulphonylalkyl radical; a C₁-C₆ alkyl(C₁-C₆)carbonylalkyl radical; a C₁-C₆ N-alkyl(C₁-C₆)carbamylalkyl radical; or a C₁-C₆ N-alkyl(C₁-C₆)sulphonamidoalkyl radical;
x is 0 or 1:
if x=0, the linking arm D is attached to the nitrogen atom;
if x=1, the linking arm D is attached to one of the vertices E, G, J or L; and
Y is a counterion.

13. The composition of claim 12, wherein the cationic tertiary paraphenylenediamine is such that the vertices E, G, J and L form an imidazole ring.

14. The composition of claim 12, wherein the cationic tertiary paraphenylenediamine is such that x is equal to 0 and D is a single bond or an alkylene chain capable of being substituted.

15. The composition of claim 2, in which the cationic tertiary paraphenylenediamine is such that R₂ is an onium radical Z of formula IV: 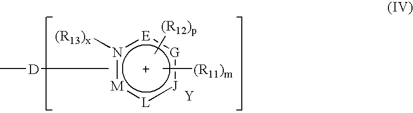
in which:
D is a single bond or a linear or branched C₁-C₁₄ alkylene chain capable of containing one or more heteroatoms selected from oxygen, sulphur and nitrogen atoms, capable of being substituted by one or more hydroxyl, C₁-C₆ alkoxy or amino radicals and capable of carrying one or more ketone functional groups;
the vertices E, G, J, L and M, which are identical or different, are a carbon, oxygen, sulphur or nitrogen atom so as to form a ring selected from pyridine, pyrimidine, pyrazine, triazine and pyridazine rings;
p is an integer between 0 and 3 inclusive;
m is an integer between 0 and 5 inclusive;
p+m is an integer between 0 and 5;
the radicals R₁₁, which are identical or different, are a halogen atom; a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a carboxyl radical; a C₁-C₆ alkylcarbonyl radical; a thio radical; a C₁-C₆ thioalkyl radical; an alkyl(C₁-C₆)thio radical; an amino radical; an amino radical substituted by an alkyl(C₁-C₆), alkyl(C₁-C₆)carbonyl, amido or alkyl(C₁-C₆)sulphonyl radical; a C₁-C₆ monohydroxyalkyl radical; or a C₂-C₆ polyhydroxyalkyl radical, it being understood that the radicals R₁₁ are carried by a carbon atom;
the radicals R₁₂, which are identical or different, are a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; a C₁-C₆ carbamylalkyl radical; a C₁-C₆ alkyl(C₁-C₆)carboxyalkyl radical; or a benzyl radical, it being understood that the radicals R₁₂ are carried by a nitrogen;
R₁₃ is a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carboxyalkyl radical; a C₁-C₆ carbamylalkyl radical; a C₁-C₆ trifluoroalkyl radical; a C₁-C₆ trialkyl(C₁-C₆)silanalkyl radical; a C₁-C₆ sulphonamidoalkyl radical; a C₁-C₆ alkyl(C₁-C₆)carboxyalkyl radical; a C₁-C₆ alkyl(C₁-C₆)sulphinylalkyl radical; a C₁-C₆ alkyl(C₁-C₆)sulphonylalkyl radical; a C₁-C₆ alkyl(C₁-C₆)carbonylalkyl radical; a C₁-C₆ N-alkyl(C₁-C₆)carbamylalkyl radical; or a C₁-C₆ N-alkyl(C₁-C₆)sulphonamidoalkyl radical;
x is 0 or 1:
if x=0, the linking arm D is attached to the nitrogen atom;
if x=1, the linking arm D is attached to one of the vertices E, G, J, L or M; and
Y is a counterion.

16. The composition of claim 15, wherein the vertices E, G, J, L and M form, with the nitrogen of the ring, a ring selected from pyridine and pyrimidine rings.

17. The composition of claim 15, wherein the cationic tertiary paraphenylenediamine is such that x is equal to 0 and R₁₁ is selected from a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a C₁-C₆ alkylcarbonyl radical; an amino radical; an amino radical monosubstituted or disubstituted by an alkyl(C₁-C₆), alkyl(C₁-C₆)carbonyl, amido or alkyl(C₁-C₆)sulphonyl radical; a C₁-C₆ monohydroxyalkyl radical; and a C₂-C₆ polyhydroxyalkyl radical; and R₁₂ is selected from a C₁-C₆ alkyl radical; a C₁-C₆ monohydroxyalkyl radical; a C₂-C₆ polyhydroxyalkyl radical; a C₁-C₆ trialkyl(C₁-C₆)silanalkyl radical; a C₁-C₆ alkoxy(C₁-C₆)alkyl radical; and a C₁-C₆ carbamylalkyl radical.

18. The composition of claim 15, wherein the cationic tertiary paraphenylenediamine is such that x is equal to 1 and R₁₃ is selected from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is monosubstituted or disubstituted by an alkyl(C₁-C₆), alkyl(C₁-C₆)carbonyl, amido or alkyl(C₁-C₆)sulphonyl radical; a C₁-C₆ carbamylalkyl radical; a C₁-C₆ trialkyl(C₁-C₆)silanalkyl radical; a C₁-C₆ alkyl(C₁-C₆)carbonylalkyl radical; and a C₁-C₆ N-alkyl(C₁-C₆)carbamylalkyl radical; R₁₁ is selected from a hydroxyl radical; a C₁-C₆ alkyl radical; a C₁-C₆ monohydroxyalkyl radical; a C₂-C₆ polyhydroxyalkyl radical; a C₁-C₆ alkoxy radical; a C₁-C₆ trialkyl(C₁-C₆)silanalkyl radical; an amido radical; a C₁-C₆ alkylcarbonyl radical; an amino radical; and an amino radical monosubstituted or disubstituted by an alkyl(C₁-C₆), alkyl(C₁-C₆)carbonyl, amido or alkyl(C₁-C₆)sulphonyl radical; and R₁₂ is selected from a C₁-C₆ alkyl radical; a C₁-C₆ monohydroxyalkyl radical; a C₂-C₆ polyhydroxyalkyl radical; a C₁-C₆ trialkyl-(C₁-C₆)silanalkyl radical; a C₁-C₆ alkoxy(C₁-C₆)alkyl radical; and a C₁-C₆ carbamylalkyl radical.

19. The composition of claim 15, wherein the cationic tertiary paraphenylenediamine is such that R₁, R₁₂ and R₁₃ are alkyl radicals capable of being substituted.

20. The composition of claim 2, wherein the cationic tertiary paraphenylenediamine is such that the radical R₂ is a radical of the formula —XP(O)(O—)OCH₂CH₂N⁺(CH₃)₃, where X is an oxygen atom or a radical —NR₁₄, R₁₄ being a hydrogen, a C₁-C₄ alkyl radical or a hydroxyalkyl radical.

21. The composition of claim 2, wherein the cationic tertiary paraphenylenediamine is such that the radical R₂ is a guanidine radical of formula —X—C═NR₈—NR₉R₁₀, where X is an oxygen atom or a radical —NR₁₁, R₈, R₉, R₁₀ and R₁₁ being a hydrogen, a C₁-C₄ alkyl radical or a hydroxyalkyl radical.

22. The composition of claim 1, wherein the cationic tertiary paraphenylenediamine is selected from the group comprising:
[1-(4-aminophenyl)pyrrolidin-3-yl]trimethylammonium; chloride;
[1-(4-aminophenyl)pyrrolidin-3-yl]dimethyltetradecylammonium; bromide;
N′-[1-(4-aminophenyl)pyrrolidin-3-yl]-N,N-dimethyl guanidinium chloride;
N-[1-(4-aminophenyl)pyrrolidin-3-yl] guanidinium chloride;
3-[1-(4-aminophenyl)pyrrolidin-3-yl]-1-methyl-3H-imidazol-1-ium; chloride;
[1-(4-aminophenyl)pyrrolidin-3-yl] (2-hydroxyethyl)dimethylammonium;
chloride; [1-(4-aminophenyl)pyrrolidin-3-yl]dimethyl(3-trimethylsilanylpropyl)ammonium; chloride;
[1-(4-aminophenyl)pyrrolidin-3-yl](trimethylammoniohexyl)dimethylammonium; dichloride;
[1-(4-aminophenyl)pyrrolidin-3-yl]oxophosphorylcholine;
{2-[1-(4-aminophenyl)pyrrolidin-3-yloxy]ethyl}trimethylammonium; chloride;
1-{2-[1-(4-aminophenyl)pyrrolidin-3-yloxy]ethyl}-1-methylpyrrolidinium; chloride;
3-{3-[1-(4-aminophenyl)pyrrolidin-3-yloxy]propyl}-1-methyl-3H-imidazol-1-ium; chloride;
1-{2-[1-(4-aminophenyl)pyrrolidin-3-yloxy]ethyl}-1-methylpiperidinium; chloride;
3-{3-[1-(5-trimethylsilanylethyl-4-amino-3-trimethylsilanylethylphenyl)pyrrolidin-3-yloxy]propyl}-1-methyl-3H-imidazol-1-ium; chloride;
[1-(4-amino-3-methylphenyl)pyrrolidin-3-yl]trimethylammonium; chloride;
[1-(4-amino-3-methylphenyl)pyrrolidin-3-yl]dimethyltetradecylammonium; chloride;
N′-[1-(4-amino-3-methylphenyl)pyrrolidin-3-yl]-N,N-dimethyl guanidinium chloride;
N-[1-(4-amino-3-methylphenyl)pyrrolidin-3-yl] guanidinium chloride;
3-[1-(4-amino-3-methylphenyl)pyrrolidin-3-yl]-1-methyl-3H-imidazol-1-ium; chloride;
[1-(4-amino-3-methylphenyl)pyrrolidin-3-yl] (2-hydroxyethyl)dimethylammonium; chloride;
[1-(4-amino-3-methylphenyl)pyrrolidin-3-yl]dimethyl(3-trimethylsilanylpropylammonium; chloride;
[1-(4-amino-3-methylphenyl)pyrrolidin-3-yl] (trimethylammoniohexyl)dimethylammonium; dichloride;
[1-(4-amino-3-methylphenyl)pyrrolidin-3-yl]oxophosphorylcholine;
{2-[1-(4-amino-3-methylphenyl)pyrrolidin-3-yloxy]ethyl}trimethylammonium; chloride;
1-{2-[1-(4-amino-3-methylphenyl)pyrrolidin-3-yloxy]ethyl}-1-methylpyrrolidinium; chloride;
3-{3-[1-(4-amino-3-methylphenyl)pyrrolidin-3-yloxy]propyl}-1-methyl-3H-imidazol-1-ium; chloride;
1-{2-[1-(4-amino-3-methylphenyl)pyrrolidin-3-yloxy]ethyl}-1-methylpiperidinium; chloride;
[1-(4-amino-3-trimethylsilanylethylphenyl)pyrrolidin-3-yl]trimethylammonium; chloride;
3-[1-(4-amino-3-trimethylsilanylethylphenyl)pyrrolidin-3-yl]-1-methyl-3H-imidazol-1-ium; chloride;
3-{3-[1-(4-amino-3-trimethylsilanylethylphenyl)pyrrolidin-3-yloxy]propyl}-1-methyl-3H-imidazol-1-ium; chloride;
[1-(5-trimethylsilanylethyl-4-amino-3-trimethylsilanylethylphenyl)pyrrolidin-3-yl]trimethylammonium; chloride;
3-[1-(5-trimethylsilanylethyl-4-amino-3-trimethylsilanylethylphenyl)pyrrolidin-3-yl]-1-methyl-3H-imidazol-1-ium; chloride;
1′-(4-aminophenyl)-1-methyl[1,3′]bipyrrolidinyl-1-ium; chloride;
1′-(4-amino-3-methylphenyl)-1-methyl[1,3′]bipyrrolidinyl-1-ium; chloride;
3-{[1-(4-aminophenyl)pyrrolidin-3-ylcarbamoyl]methyl}-1-methyl-3H-imidazol-1-ium; chloride;
3-{[1-(4-amino-3-methylphenyl)pyrrolidin-3-ylcarbamoyl]methyl}-1-methyl-3H-imidazol-1-ium; chloride;
3-[1-(4-aminophenyl)pyrrolidin-3-yl]-1-(3-trimethylsilanylpropyl)-3H-imidazol-1-ium; chloride;
3-[1-(4-aminophenyl)pyrrolidin-3-yl]-1-(3-trimethylsilanylpropyl)-3H-imidazol-1-ium; chloride;
[1-(4-aminophenyl)pyrrolidin-3-yl]ethyldimethylammonium; chloride;
[1-(4-aminophenyl)pyrrolidin-3-yl]ethyldimethylammonium; iodide;
[1-(4-aminophenyl)pyrrolidin-3-yl]propyldimethylammonium; iodide; [1-(4-aminophenyl)pyrrolidin-3-yl]propyldimethylammonium; bromide; [1-(4-aminophenyl)pyrrolidin-3-yl]propyldimethylammonium; methosulfate;
[1-(4-aminophenyl)pyrrolidin-3-yl]butyldimethylammonium; iodide;
[1-(4-aminophenyl)pyrrolidin-3-yl]pentyldimethylammonium; iodide;
[1-(4-aminophenyl)pyrrolidin-3-yl]hexyldimethylammonium; iodide;
[1-(4-aminophenyl)pyrrolidin-3-yl]heptyldimethylammonium; iodide;
[1-(4-aminophenyl)pyrrolidin-3-yl]octyldimethylammonium; iodide;
[1-(4-aminophenyl)pyrrolidin-3-yl]decyldimethylammonium; iodide;
[1-(4-aminophenyl)pyrrolidin-3-yl]hexadecyldimethylammonium; iodide;
[1-(4-aminophenyl)pyrrolidin-3-yl]hydroxyethyldimethylammonium; chloride; and
[1-(4-aminophenyl)pyrrolidin-3-yl]hydroxyethyldimethylammonium; iodide.

23. The composition of claim 1, wherein the cationic tertiary paraphenylenediamine is selected from the group comprising:
[1-(4-aminophenyl)pyrrolidin-3-yl]trimethylammonium; chloride;
[1-(4-aminophenyl)pyrrolidin-3-yl]dimethyltetradecylammonium; bromide;
N′-[1-(4-aminophenyl)pyrrolidin-3-yl]-N,N-dimethyl guanidinium chloride;
N-[1-(4-aminophenyl)pyrrolidin-3-yl]guanidinium chloride;
3-[1-(4-aminophenyl)pyrrolidin-3-yl]-1-methyl-3H-imidazol-1-ium; chloride;
[1-(4-aminophenyl)pyrrolidin-3-yl] (2-hydroxyethyl)dimethylammonium; chloride;
[1-(4-aminophenyl)pyrrolidin-3-yl]dimethyl(3-trimethylsilanylpropyl)ammonium; chloride;
[1-(4-amino-3-methylphenyl)pyrrolidin-3-yl]trimethylammonium; chloride;
[1-(4-amino-3-methylphenyl)pyrrolidin-3-yl]dimethyltetradecyl-ammonium; chloride;
N′-[1-(4-amino-3-methylphenyl)pyrrolidin-3-yl]-N,N-dimethyl guanidinium chloride;
N-[1-(4-amino-3-methylphenyl)pyrrolidin-3-yl] guanidinium chloride;
3-[1-(4-amino-3-methylphenyl)pyrrolidin-3-yl]-1-methyl-3H-imidazol-1-ium; chloride;
[1-(4-amino-3-methylphenyl)pyrrolidin-3-yl](2-hydroxyethyl)dimethylammonium; chloride;
[1-(4-amino-3-methylphenyl)pyrrolidin-3-yl]dimethyl(3-trimethylsilanylpropylammonium; chloride;
1′-(4-aminophenyl)-1-methyl [1,3′]bipyrrolidinyl-1-ium; chloride;
1′-(4-amino-3-methylphenyl)-1-methyl[1,3′]bipyrrolidinyl-1-ium; chloride;
3-{[1-(4-aminophenyl)pyrrolidin-3-ylcarbamoyl]methyl}-1-methyl-3H-imidazol-1-ium; chloride;
3-{[1-(4-amino-3-methylphenyl)pyrrolidin-3-ylcarbamoyl]methyl}-1-methyl-3H-imidazol-1-ium; chloride;
3-[1-(4-aminophenyl)pyrrolidin-3-yl]-1-(3-trimethylsilanylpropyl)-3H-imidazol-1-ium; chloride;
3-[1-(4-amino-3-methylphenyl)pyrrolidin-3-yl]-1-(3-trimethylsilanylpropyl)-3H-imidazol-1-ium; chloride;
[1-(4-aminophenyl)pyrrolidin-3-yl]ethyldimethylammonium; chloride;
[1-(4-aminophenyl)pyrrolidin-3-yl]ethyldimethylammonium; iodide;
[1-(4-aminophenyl)pyrrolidin-3-yl]propyldimethylammonium; iodide;
[1-(4-aminophenyl)pyrrolidin-3-yl]propyldimethylammonium; bromide;
[1-(4-aminophenyl)pyrrolidin-3-yl]propyldimethylammonium; methosulfate;
[1-(4-aminophenyl)pyrrolidin-3-yl]butyldimethylammonium; iodide;
[1-(4-aminophenyl)pyrrolidin-3-yl]pentyldimethylammonium; iodide;
[1-(4-aminophenyl)pyrrolidin-3-yl]hexyldimethylammonium; iodide;
[1-(4-aminophenyl)pyrrolidine-3-yl]heptyldimethylammonium; iodide;
[1-(4-aminophenyl)pyrrolidin-3-yl]octyldimethylammonium; iodide;
[1-(4-aminophenyl)pyrrolidin-3-yl]decyldimethylammonium; iodide;
[1-(4-aminophenyl)pyrrolidin-3-yl]hexadecyldimethylammonium; iodide;
[1-(4-aminophenyl)pyrrolidin-3-yl]hydroxyethyldimethylammonium; chloride; and
[1-(4-aminophenyl)pyrrolidin-3-yl]hydroxyethyldimethylammonium; iodide.

24. The composition of claim 1, wherein the cationic tertiary paraphenylenediamine is selected from the group comprising:
[1-(4-aminophenyl)pyrrolidin-3-yl]trimethylammonium; chloride;
[1-(4-aminophenyl)pyrrolidin-3-yl]dimethyltetradecylammonium; bromide;
N′-[1-(4-aminophenyl)pyrrolidin-3-yl]-N,N-dimethyl guanidinium chloride;
N-[1-(4-aminophenyl)pyrrolidin-3-yl] guanidinium chloride;
3-[1-(4-aminophenyl)pyrrolidin-3-yl]-1-methyl-3H-imidazol-1-ium; chloride;
[1-(4-aminophenyl)pyrrolidin-3-yl](2-hydroxyethyl)dimethylammonium; chloride;
[1-(4-aminophenyl)pyrrolidin-3-yl]dimethyl(3-trimethylsilanylpropyl)ammonium; chloride;
[1-(4-aminophenyl)pyrrolidin-3-yl] (trimethylammoniohexyl)dimethylammonium; dichloride;
1′-(4-aminophenyl)-1-methyl[1,3′]bipyrrolidinyl-1-ium; chloride;
3-[1-(4-aminophenyl)pyrrolidin-3-yl]-1-(3-trimethylsilanylpropyl)-3H-imidazol-1-ium; chloride;
3-[1-(4-amino-3-methylphenyl)pyrrolidin-3-yl]-1-(3-trimethylsilanyl-propyl)-3H-imidazol-1-ium; chloride;
[1-(4-aminophenyl)pyrrolidin-3-yl]ethyldimethylammonium; chloride;
[1-(4-aminophenyl)pyrrolidin-3-yl]ethyldimethylammonium; iodide;
[1-(4-aminophenyl)pyrrolidin-3-yl]propyldimethylammonium; iodide;
[1-(4-aminophenyl)pyrrolidin-3-yl]propyldimethylammonium; bromide;
[1-(4-aminophenyl)pyrrolidin-3-yl]propyldimethylammonium; methosulphate;
[1-(4-aminophenyl)pyrrolidin-3-yl]butyldimethylammonium; iodide;
[1-(4-aminophenyl)pyrrolidin-3-yl]pentyldimethylammonium; iodide;
[1-(4-aminophenyl)pyrrolidin-3-yl]hexyldimethylammonium; iodide;
[1-(4-aminophenyl)pyrrolidin-3-yl]heptyldimethylammonium; iodide;
[1-(4-aminophenyl)pyrrolidin-3-yl]octyldimethylammonium; iodide;
[1-(4-aminophenyl)pyrrolidin-3-yl]decyldimethylammonium; iodide;
[1-(4-aminophenyl)pyrrolidin-3-yl]hexadecyldimethylammonium; iodide;
[1-(4-aminophenyl)pyrrolidin-3-yl]hydroxyethyldimethylammonium; chloride; and
[1-(4-aminophenyl)pyrrolidin-3-yl]hydroxyethyldimethylammonium; iodide.

25. The composition of claim 1, wherein the cationic tertiary paraphenylenediamine is selected from the group comprising:
[1-(4-aminophenyl)pyrrolidin-3-yl]trimethylammonium; chloride;
3-[1-(4-aminophenyl)pyrrolidin-3-yl]-1-methyl-3H-imidazol-1-ium; chloride;
[1-(4-aminophenyl)pyrrolidin-3-yl](2-hydroxyethyl)dimethylammonium; chloride; and
1′-(4-aminophenyl)-1-methyl[1,3′]bipyrrolidinyl-1-ium; chloride.

26. The composition of claim 1, wherein the cationic tertiary paraphenylenediamine is selected from the group comprising:
[1-(4-aminophenyl)pyrrolidin-3-yl]trimethylammonium; chloride; and
[1-(4-aminophenyl)pyrrolidin-3-yl](2-hydroxyethyl)dimethylammonium; chloride.

27. The composition of claim 1, wherein the monosaccharide is selected from aldoses and ketoses.

28. The composition of claim 27, wherein the monosaccharide is selected from tetroses, pentoses, hexoses and heptoses.

29. The composition of claim 28, wherein the hexoses and heptoses are selected from furanoses and pyranoses.

30. The composition of claim 27, wherein the monosaccharides are selected from erythrose, threose, ribose, arabinose, xylose, lyxose, allose, altrose, glucose, mannose, gulose, idose, galactose and talose.

31. The composition of claim 27, wherein the monosaccharide is selected from α-D-glucopyranose and β-D-glucopyranose.

32. The composition of claim 27, wherein the monosaccharides are selected from erythrulose, ribulose, xylulose, psicose, fructose, sorbose and tagatose.

33. The composition of claim 1, wherein the disaccharide contains 10, 11 or 12 carbon atoms.

34. The composition of claim 33, wherein the disaccharide is selected from sucrose, lactose and maltose.

35. The composition of claim 1, wherein the cationic tertiary paraphenylenediamine(s) with a pyrrolidine ring represent from 0.001 to 10% and preferably from 0.005 to 6% by weight, based on the total weight of the composition.

36. The composition of claim 1, wherein the monosaccharide(s) and/or disaccharide(s) represent from 0.01 to 20% and preferably from 0.05 to 5% by weight, based on the total weight of the composition.

37. The composition of claim 1, wherein the composition further comprises at least one cationic polymer.

38. The composition of claim 1, wherein the composition further comprises at least one thickening polymer.

39. The composition of claim 1, wherein the composition further comprises at least one surfactant selected from the group comprising anionic surfactants, amphoteric or zwitterionic surfactants, non-ionic surfactants and cationic surfactants.

40. The composition of claim 1, wherein the composition further comprises at least one additional oxidation base other than the cationic tertiary paraphenylenediamines with a pyrrolidine ring, selected from paraphenylenediamines, bis-phenylalkylenediamines, paraminophenols, orthoaminophenols, heterocyclic bases and their addition salts.

41. The composition of claim 40, wherein the additional oxidation base(s) are present in an amount of between 0.001 and 20% by weight and preferably of between 0.005 and 6% by weight, based on the total weight of the composition.

42. The composition of claim 1, wherein the composition further comprises at least one coupler selected from metaphenylenediamines, metaminophenols, metadiphenols, naphthalene couplers, heterocyclic couplers and their addition salts.

43. The composition of claim 42, wherein the coupler is selected from 1,3-dihydroxybenzene, 1,3-dihydroxy-2-methylbenzene, 4-chloro-1,3-dihydroxybenzene, 2,4-diamino-1-(β-hydroxyethoxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-B-hydroxyethylamino-3,4-methylenedioxybenzene, c-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(13-hydroxyethylamino)toluene and their addition salts.

44. The composition of claim 42, wherein the coupler(s) are present in an amount of between 0.001 and 20% and preferably of between 0.005 and 6% by weight, based on the total weight of the composition.

45. The composition of claim 1, wherein the composition further comprises at least one direct dyestuff.

46. The composition of claim 1, wherein the composition further comprises at least one hydroxylated solvent such as ethanol, propylene glycol, glycerol or a polyol monoether.

47. The composition of claim 1, wherein the composition further comprises an oxidizing agent selected from hydrogen peroxide, urea peroxide, alkali metal bromates, per-salts, per-acids and oxidase enzymes, hydrogen peroxide being preferred.

48. A process for the oxidation dyeing of keratin fibres, wherein a dyeing composition as defined in claim 1 is applied to the fibres in the presence of an oxidizing agent.

49. A multicompartment device, wherein a first compartment comprises a composition for dyeing keratin fibres, as defined in claim 1, and a second compartment contains an oxidizing agent.
